(19) United States
(12) Patent Application Publication
(10) Pub. No.: US 2023/0110128 A1
(43) Pub. Date: Apr. 13, 2023

(54) USE OF ANTIBODY DRUG CONJUGATES COMPRISING TUBULIN DISRUPTING AGENTS TO TREAT SOLID TUMOR

(71) Applicant: Seagen Inc., Bothell, WA (US)

(72) Inventors: Anthony Toa CAO, Sammamish, WA (US); Shyra Jane GARDAI, Monroe, WA (US)

(21) Appl. No.: 17/870,749

(22) Filed: Jul. 21, 2022

Related U.S. Application Data
(63) Continuation of application No. 16/362,125, filed on Mar. 22, 2019, now abandoned.
(60) Provisional application No. 62/658,276, filed on Apr. 16, 2018.
(60) Provisional application No. 62/647,346, filed on Mar. 23, 2018.

(51) Int. Cl. A61K 47/68; C07K 16/08; C07K 16/30; C07K 16/28; C07K 16/32; A61K 31/4375; A61P 35/00; C07K 16/22; A61K 31/165; A61K 31/22; A61K 47/65; C07K 16/24; A61K 31/337; A61K 39/00

(52) U.S. Cl. CPC A61K 47/6805; A61K 47/6803; C07K 16/087; C07K 16/3084; C07K 16/2809; C07K 16/2893; C07K 16/32; A61K 31/4375; C07K 16/2818; A61P 35/00; C07K 16/2827; C07K 16/2887; A61K 47/6849; A61K 47/6889; C07K 16/2881; C07K 16/22; C07K 16/2878; A61K 31/165; A61K 31/22; A61K 47/65; C07K 16/241; A61K 31/337; C07K 16/248; A61K 47/6851; C07K 16/2896; C07K 16/2875; A61K 2039/505

(57) ABSTRACT

The present disclosure, relates, in general to methods for treating solid tumors comprising administering a drug-linker-antibody conjugate, wherein the drug is a tubulin disrupting agent.

### CROSS REFERENCE TO RELATED APPLICATIONS

The present application is a continuation of U.S. patent Ser. No. 16/362,125, filed Mar. 22, 2019, now abandoned, which claims the priority benefit of U.S. Provisional Patent Application No. 62/647,346, filed Mar. 23, 2018, and U.S. Provisional Patent Application No. 62/658,276, filed Apr. 16, 2018, the contents of which are herein incorporated by reference in their entireties.

### REFERENCE TO AN ELECTRONIC SEQUENCE LISTING

The content of the electronic sequence listing (761682004501SEQLIST.xml; Size: 16,616 bytes; and Date of Creation: Oct. 3, 2022) is herein incorporated by reference in its entirety.

### FIELD OF THE DISCLOSURE

The present disclosure relates, in general, to methods of treating a solid tumor comprising administering a Drug-Linker Unit-Antibody conjugate therapy, wherein the drug is a tubulin disrupting agent.

### BACKGROUND

Microtubules are important heterodimeric structures involved in many cell processes such as cell division and cell transport. Disruption of microtubules induces cell cycle arrest in the G2/M phase. Microtubule/tubulin inhibitors can be classified into two major categories according to their mechanisms of action: agents promoting tubulin polymerization and stabilizing microtubule structures (e.g., paclitaxel) and agents inhibiting tubulin polymerization and destabilizing microtubule structures (such as maytansinoids, auristatins, vinblastine and vincristine) (Chen et al., Molecules 22:1281, 2017).

Tubulin disrupting agents such as MMAE have been used on antibody drug conjugates for leukemia. For example, Brentuximab vedotin is an antibody-drug conjugate composed of an anti-CD30 monoclonal antibody conjugated by a protease-cleavable linker to the microtubule disrupting agent, monomethyl auristatin E. Brentuximab vedotin has been approved for the treatment of classical Hodgkin lymphoma patients after failure of autologous stem cell transplant (ASCT) or after failure of at least 2 prior multi-agent chemotherapy regimens in patients who are not ASCT candidates, and as consolidation post-ASCT for Hodgkin lymphoma patients at increased risk of relapse/progression. See ADCETRIS® (brentuximab vedotin) US Prescribing Information and ADCETRIS® (brentuximab vedotin) EU Summary of Product Characteristics. It has also been approved for systemic anaplastic large cell lymphoma after failure of at least one prior multi-agent chemotherapy regimen. The anti-CD30 MMAE ADC has not been shown to be effective in solid tumors.

### SUMMARY

The present disclosure provides improved methods for treating solid tumors comprising administering an antibody drug conjugate comprising a tubulin disrupting agent. It is disclosed herein that tubulin disrupting agents effect ER stress protein pathways in solid tumor cells and induce ATP secretion and other ER stress phenomena that induce immune cell to migrate to the tumor site and reduce tumor growth.

Provided herein is a method for treating a solid tumor comprising administering to a subject in need thereof an antibody drug conjugate agent having the formula Drug-Linker Unit-Antibody (D-LU-Ab), wherein D is a tubulin disrupting agent, in an amount effective to treat the solid tumor.

Also provided is a method for modulating ATP release in a solid tumor comprising administering an antibody drug conjugate agent having the formula Drug-Linker Unit-Antibody (D-LU-Ab), wherein D is a tubulin disrupting agent, in an amount effective to induce apoptosis in the solid tumor.

Further contemplated by the disclosure is a method of inducing immune cell migration to a solid tumor comprising administering to a subject in need thereof an antibody drug conjugate agent having the formula Drug-Linker Unit-Antibody (D-LU-Ab), wherein D is a tubulin disrupting agent, in an amount effective to induce immune cell infiltration into the solid tumor.

In another aspect, the disclosure provides a method for inducing immunogenic cell death (ICD) in a solid tumor comprising administering to a subject in need thereof an antibody drug conjugate agent having the formula Drug-Linker Unit-Antibody (D-LU-Ab), wherein D is a tubulin disrupting agent, in an amount effective to induce immunogenic cell death in the solid tumor.

It is understood that the Drug-Linker Unit-Antibody (D-LU-Ab) may also be referred to herein as an antibody drug conjugate or ADC.

In various embodiments, the antibody binds to an antigen on the surface of a cancer cell. In various embodiments, the antibody is specific for CD30, CD19, CD70, CD71, CD20, CD52, CD133, EGFR, HER2, VEGF, VEGFR2, PD-1, PDL1, RANKL, CTLA-4, IL-6, SLAMF7, CD3, TNF-alpha, PDGFR-alpha, CD38, GD2, cCLB8, p97, Nectin-4, or EpCAM.

In various embodiments, the tubulin-disrupting agent increases ER stress protein pathways, increases ATP secretion and increases High mobility group box 1 (HMGB1) protein.

In various embodiments, the tubulin disrupting agent is selected from the group consisting of an auristatin, a tubulysin, a colchicine, a vinca alkaloid, a taxane, a cryptophycin, a maytansinoid, a hemiasterlin, and other tubulin disrupting agents. Exemplary tubulin disrupting agents contemplated for use in the present methods are described in greater detail in the Detailed Description.

In various embodiments, the tubulin disrupting agent is an auristatin selected from the group consisting of monomethyl auristatin E (MMAE) monomethyl auristatin F (MMAF), and dolostatin-10.

In various embodiments, the tubulin disrupting agent is a tubulysin selected from the group consisting of tubulysin D, tubuphenylalanine and tubutyrosine.

In various embodiments, the tubulin disrupting agent is a colchicine selected from the group consisting of colchicine and CA-4.

In various embodiments, the tubulin disrupting agent is a vinca alkaloid selected from the group consisting of Vinblastine (VBL), vinorelbine (VRL), vincristine (VCR) and vindesine (VDS).

In various embodiments, the tubulin disrupting agent is a taxane selected from the group consisting of paclitaxel and docetaxel.

In various embodiments, the tubulin disrupting agent is a cryptophycin selected from the group consisting of cryptophycin-1 and cryptophycin-52

In various embodiments, the tubulin disrupting agent is a maytansinoid selected from the group consisting of maytansine, maytansinol, maytansine analogs, DM1, DM3 and DM4, and ansamatocin-2.

In various embodiments, the tubulin disrupting agent is an hemiasterlin selected from the group consisting of hemiasterlin and HTI-286.

In various embodiments, the tubulin disrupting agent is selected from the group consisting of taccalonolide A, taccalonolide B, taccalonolide AF, taccalonolide AJ, taccalonolide AI-epoxide, discodermolide, epothilone A, epothilone B, and laulimalide.

In various embodiments, the solid tumor is selected from the group consisting of lung cancer, breast cancer, ovarian cancer, cervical cancer, gastrointestinal cancers, head and neck cancer, melanoma, sarcoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm, renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non small cell lung cancer (NSCLC), squamous cell non-small cell lung cancer, breast cancer, recurrent metastatic breast cancer, hepatocellular carcinoma, richter's syndrome; waldenstrom macroglobulinemia, adult glioblastoma; adult gliosarcoma, recurrent glioblastoma, recurrent childhood rhabdomyosarcoma, recurrent ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and Sezary syndrome. In one embodiment, the solid tumor is a non-lymphoma solid tumor. In some embodiments, the solid tumor may be multiple myeloma.

In various embodiments, the Drug-Linker Unit-Antibody conjugate/antibody drug conjugate comprises a protease cleavable linker, an acid-cleavable linker or a disulfide linker.

In various embodiments, the protease cleavable linker comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In various embodiments, the acid cleavable linker is a hydrazine linker or a quaternary ammonium linker.

In various embodiments, the method further comprises administering a chemotherapy regimen to the subject.

In various embodiments, the chemotherapy regimen consists essentially of doxorubicin, vinblastine, and dacarbazine (AVD) as a combination therapy. In other embodiments, the chemotherapy regimen consists essentially of cyclophosphamide, vincristine and prednisone (CHP) as a combination therapy.

In various embodiments, the antibody of the antibody drug conjugate is a monoclonal antibody. In various embodiments, the antibody is a human or humanized antibody.

In various embodiments, the antibody is an anti-CD30 antibody and the anti-CD30 antibody drug conjugate comprises i) a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR13 set out in SEQ ID NO: 16.

In certain embodiments, the antibody is an anti-CD30 antibody and the anti-CD30 antibody drug conjugate comprises i) an amino acid sequence at least 85% identical to a heavy chain variable region set out in SEQ ID NO: 2, and ii) an amino acid sequence at least 85% identical to a light chain variable region set out in SEQ ID NO: 10. It is contemplated that the amino acid variable region sequence can be 90%, 95%, 96% 97%, 98% or 99% identical to either SEQ ID NO: 2 or SEQ ID NO: 10.

In various embodiments, the antibody is an anti-CD30 antibody and the anti-CD30 antibody of the antibody drug conjugate is a chimeric AC10 antibody.

In various embodiments, the Drug-Linker Unit-Antibody conjugate/antibody drug conjugate comprises monomethyl auristatin E and a protease-cleavable linker. In various embodiments, the protease cleavable linker comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In various embodiments, the anti-CD30 antibody drug conjugate is brentuximab vedotin. In various embodiments, the anti-CD30 antibody drug conjugate is administered every 3 weeks.

In various embodiments, the anti-CD30 antibody of the anti-CD30 antibody drug conjugate is a monoclonal anti-CD30 antibody. In various embodiments, the anti-CD30 antibody of the anti-CD30 antibody drug conjugate is a chimeric AC10 antibody.

In various embodiments, the antibody drug conjugate comprises monomethyl auristatin E and a protease-cleavable linker. In various embodiments, the protease cleavable linker is comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In various embodiments, the antibody is an IgG antibody, preferably an IgG1 or IgG2 antibody.

It is understood that each feature or embodiment, or combination, described herein is a nonlimiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIGS. 1A-1C show levels of ER stress protein induction after treatment with MMAE. Western blots indicate levels of protein and phosphorylation (FIG. 1A). FIG. 1B shows levels of ATP secretion and FIG. 10 shows levels of HMGB1 release from cells.

FIGS. 2A-28 illustrate levels of ER stress induction for tubulin disrupting agents MMAE, vincristine and Paclitaxel. FIG. 2A shows ER stress induction by a CHOP luciferase assays and FIG. 2B shows ER stress induction in a xenograft model in vivo.

FIGS. 3A-3E shows ATP secretion and other effects in response to tubulin disrupting agents MMAE, vincristine and Paclitaxel MiaPac2 pancreatic cells (FIG. 3A, ATP) or PC-3 prostate tumor cells (FIG. 3B, ATP). Treatment of PC-3 cells with MMAE elicits ER stress (phosphorylation of IRE1 and INK) (FIG. 3C), release of ATP (FIG. 3D) and HMGB1 release (FIG. 3E).

FIGS. 4A-D show the effects of treatment on engrafted PC-3 cells and immune cell infiltration in athymic nude mice. FIG. 4A, dendritic cells; FIG. 4B, macrophage infiltration;

FIG. 40, dendritic cell antigen presentation; FIG. 4D, macrophage antigen presentation.

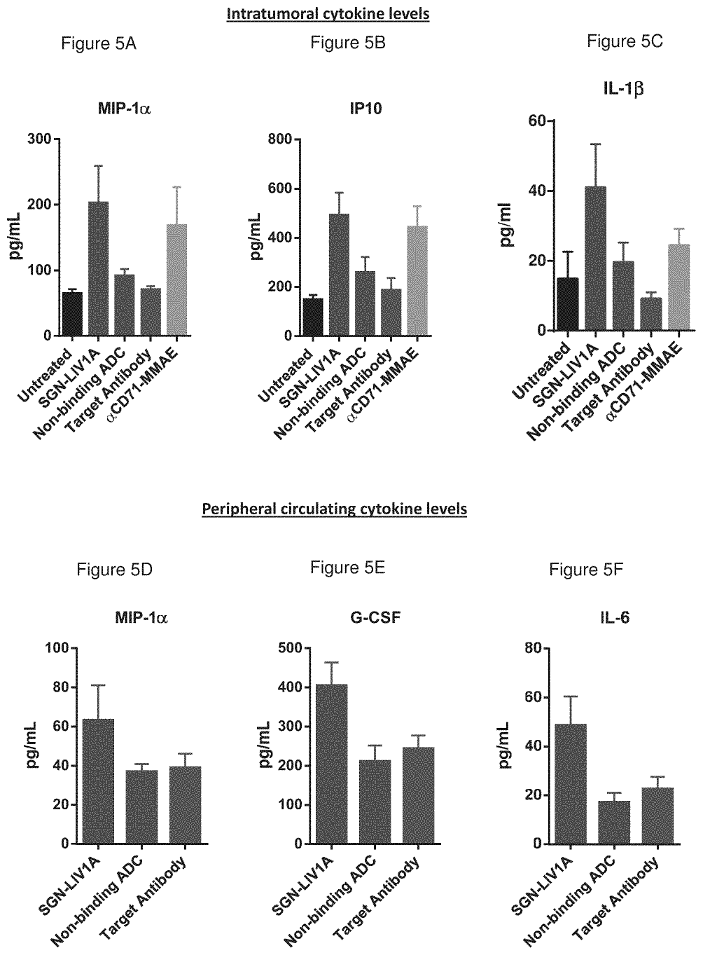

FIGS. 5A-E show the effects of treatment on cytokine/chemokine production as measured by ELISA on engrafted PC-3 cells in athymic nude mice. FIGS. 5A-50, intratumoral cytokine levels of MIP-1a, IP-10 and IL-1B, respectively; FIGS. 5D-5F, peripheral circulating cytokine levels IP-10 GCSF, and IL-6, respectively

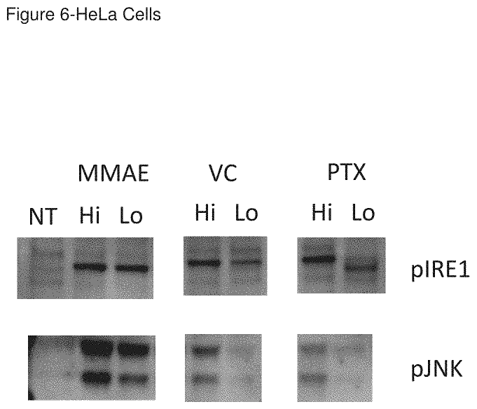

FIG. 6 shows ER stress induction by Western blot of HeLa cervical cancer cells after treatment with MMAE vincristine and Paclitaxel.

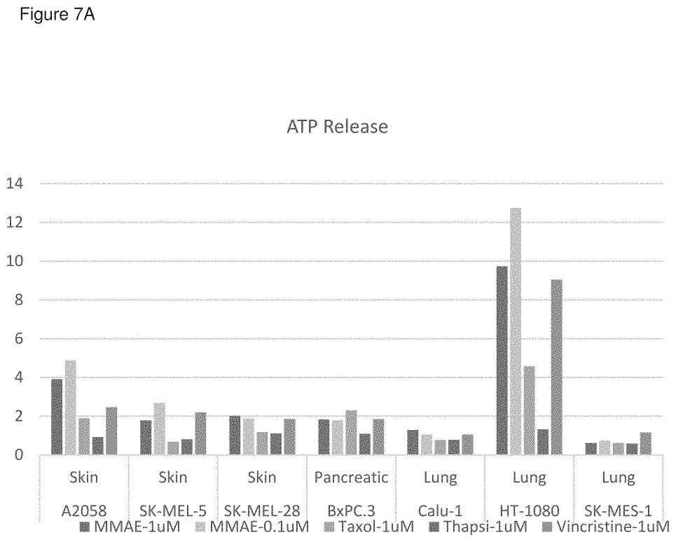

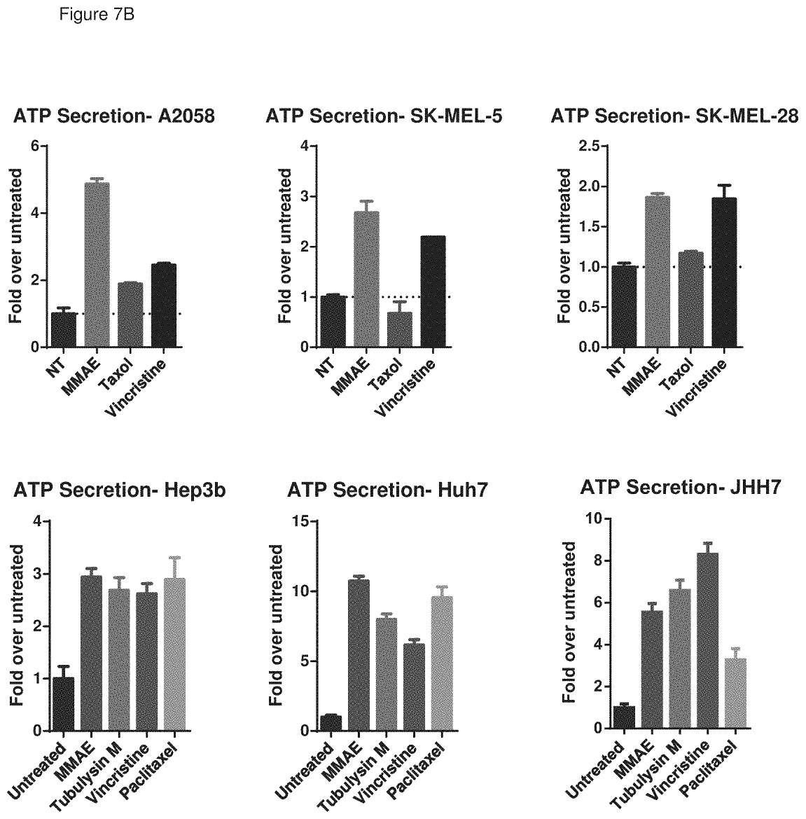

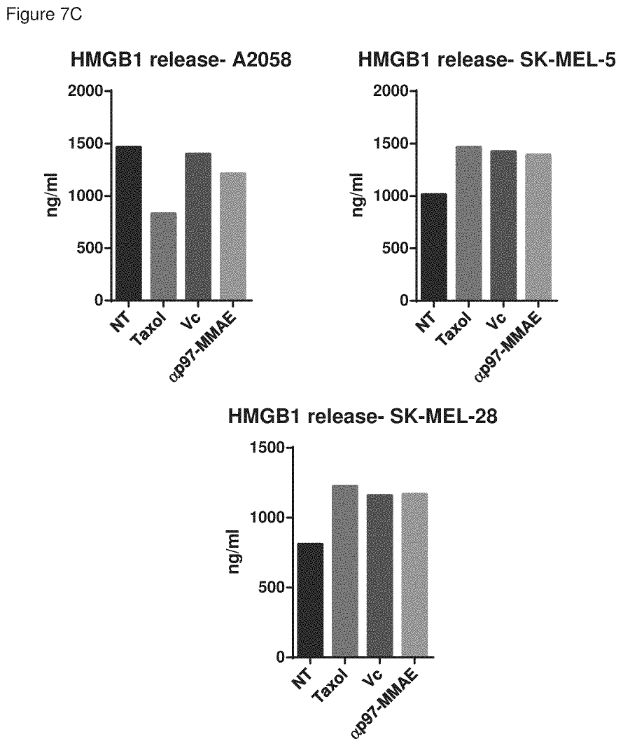

FIGS. 7A and 7B show the effects of treatment with MMAE, vincristine and Paclitaxel in skin cell solid tumor lines on ATP release as a group (FIG. 7A) and broken down by cell type (FIG. 7B). FIG. 7C shows the effects of treatment on HMGB1 release in skin cancer cells.

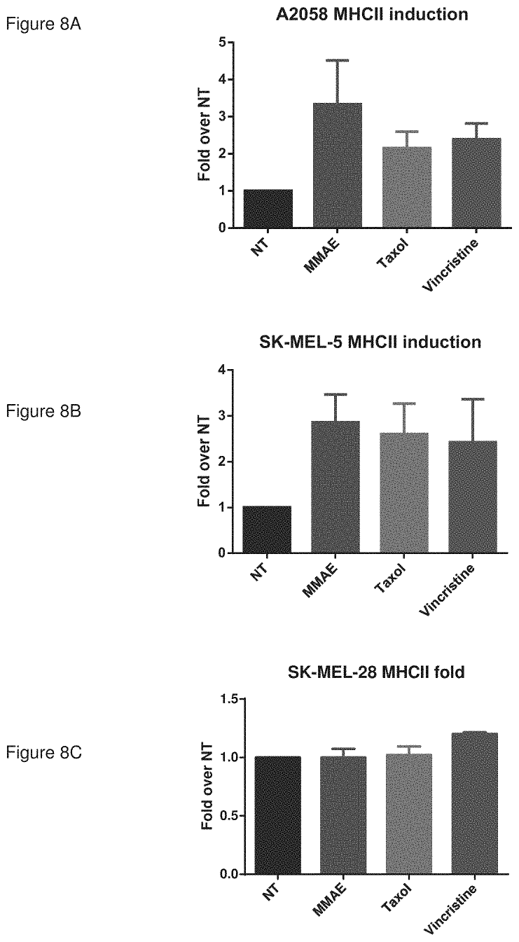

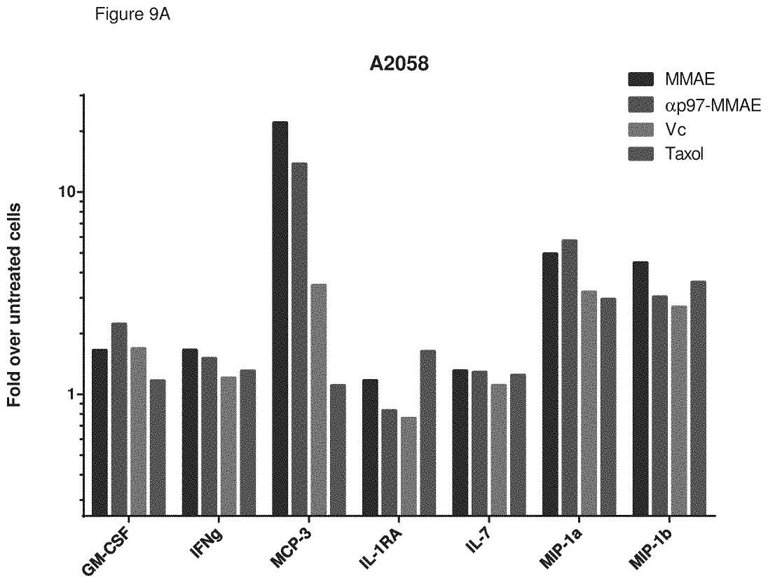

FIGS. 8A-8C show MMAE treatment of A2058 (FIG. 8A), SK-MEL-5 (FIG. 8B), and SK-MEL-28 (FIG. 8C) skin cells led to the increase in antigen-presentation in 2/3 tumor cell lines that was more robust than Paclitaxel.

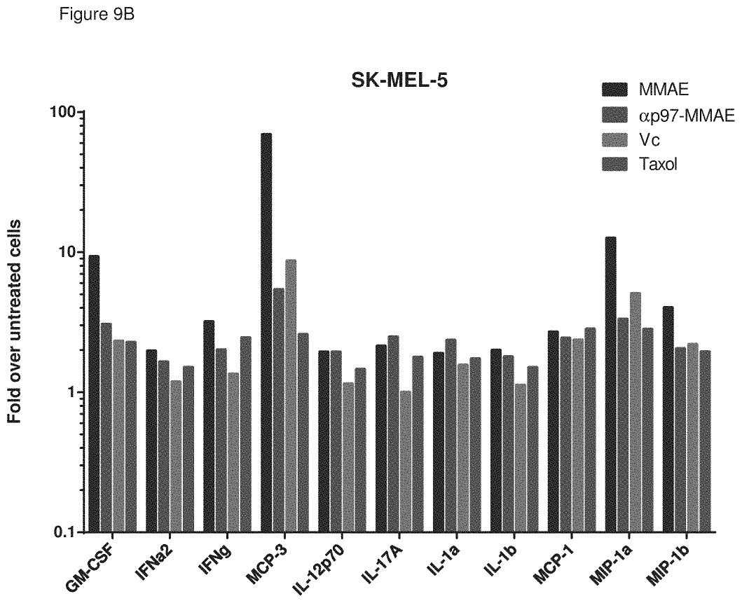

FIGS. 9A-9B show the effects of treatment of A2058 (FIG. 9A) or SK-MEL-5 (FIG. 9B) tumor cells with MMAE, vincristine, Paclitaxel or anti-p97-MMAE on the tested cytokines and chemokines.

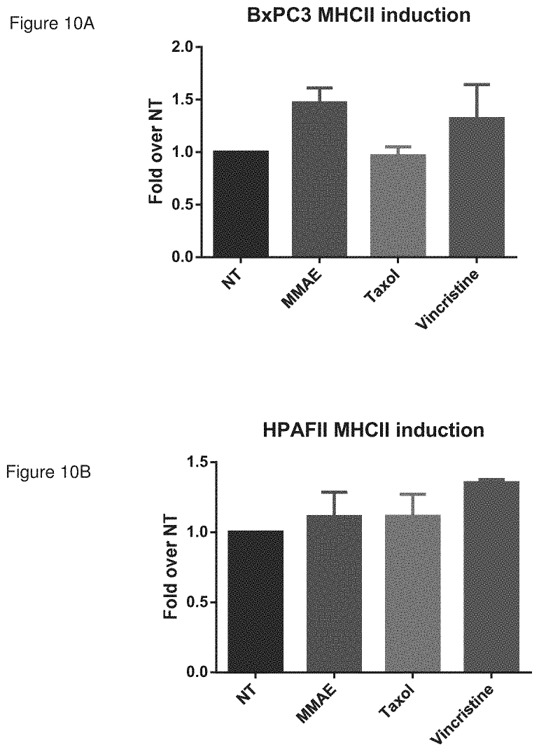

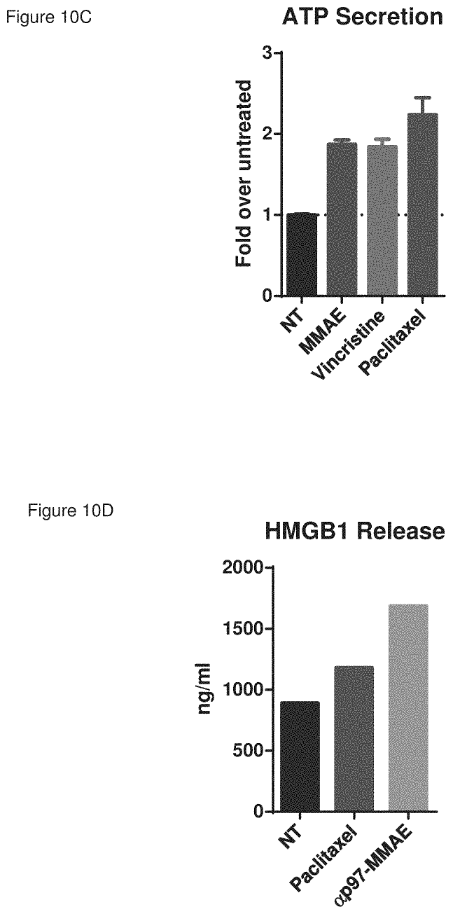

FIGS. 10A-10B show the effects of MMAE, vincristine, and Paclitaxel on increase in antigen presentation of BxPC3 (FIG. 10A) and HPAFII (FIG. 10B) cells. FIGS. 100-10D show ATP secretion and HMGB-1 release, respectively, in BxPC-3 cells.

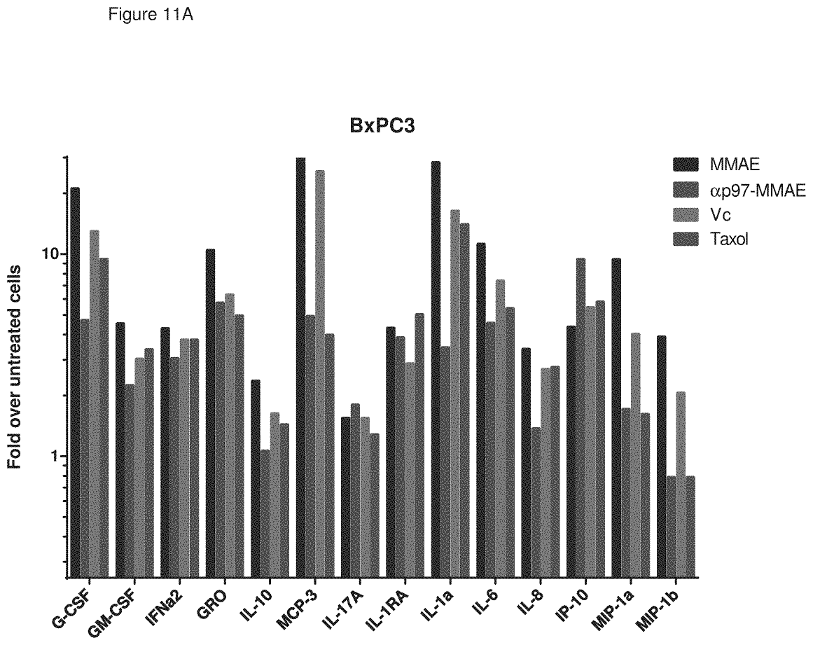

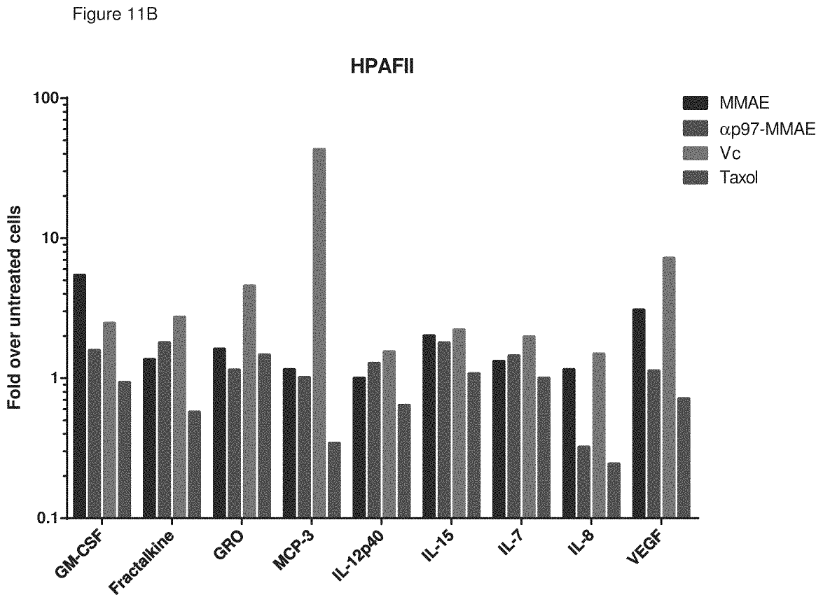

FIGS. 11A-11B show the effects of treatment of BxPC3 (FIG. 11A) or HPAFII (FIG. 11B) tumor cells with MMAE, vincristine, Paclitaxel or anti-p97-MMAE on the tested cytokines and chemokines

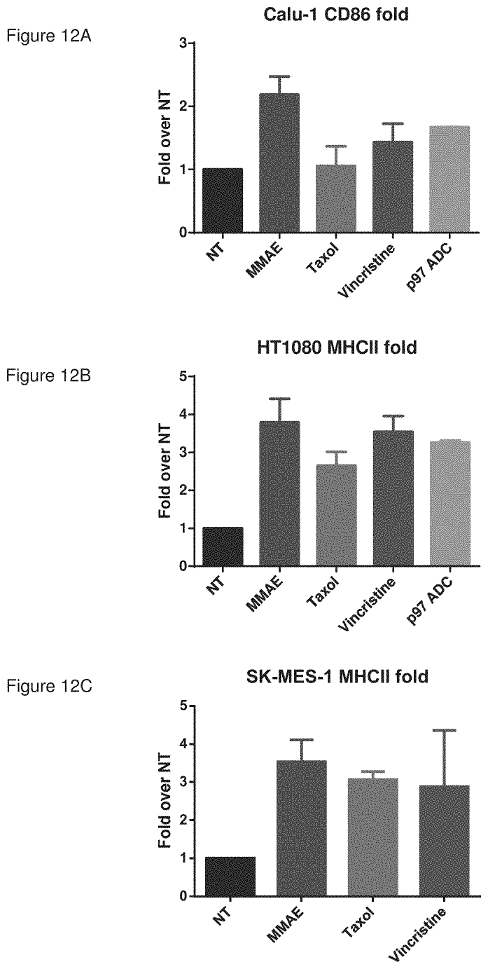

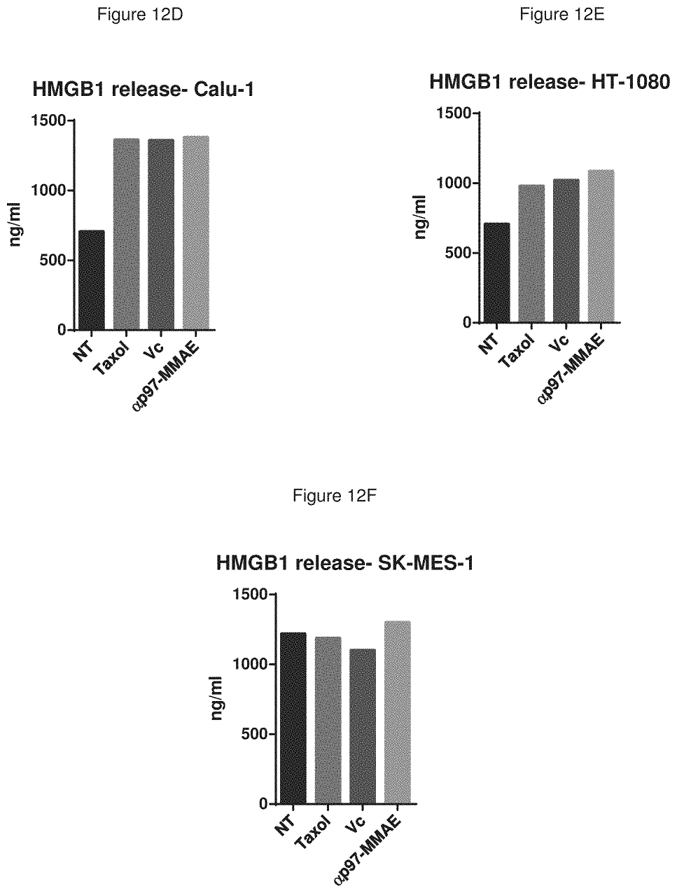

FIGS. 12A-120 show the effects of MMAE, vincristine, Paclitaxel or p97-MMAE treatment of Calu-1 (FIG. 12A), HT1080 (FIG. 12B) and SK-MES-1 (FIG. 120) cells on levels of antigen presentation after co-culture with macrophages. FIGS. 12D-12F show HMGB-1 release for Calu-1 (FIG. 12D), HT-1080 (FIG. 12E) and SK-MES-1 cells (FIG. 12F).

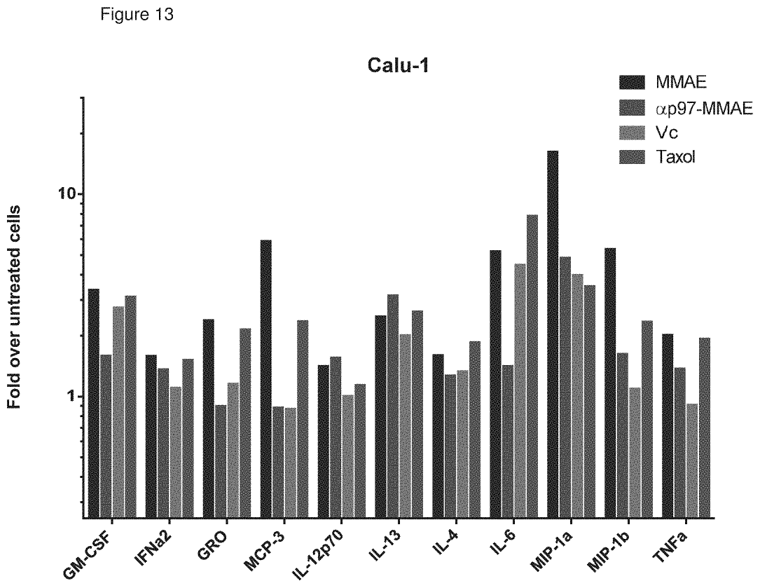

FIG. 13 shows the levels of cytokine or chemokine induction in Calu-1 cells after treatment with MMAE, vincristine, Paclitaxel or p97-MMAE.

FIGS. 14A-14B show the effects of MMAE, an MMAE-containing ADC (anti-CD71 OKT9), vincristine, and Paclitaxel on MCF7 cell antigen presentation (FIG. 14A) and cytokine/chemokine production (FIG. 148).

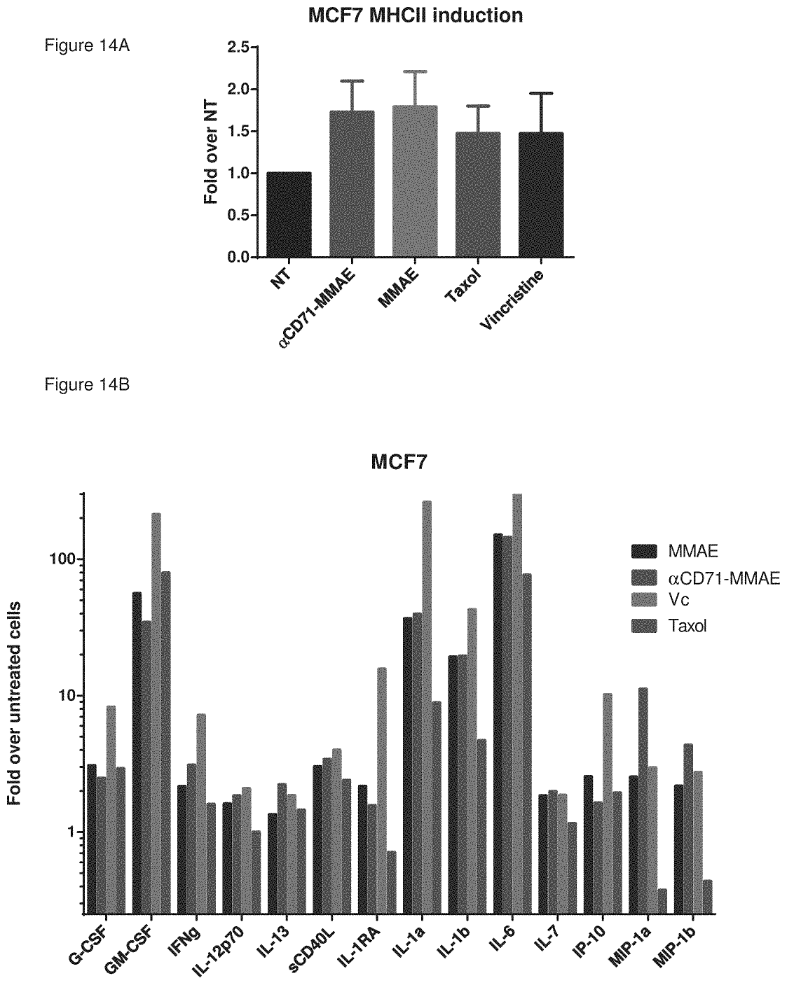

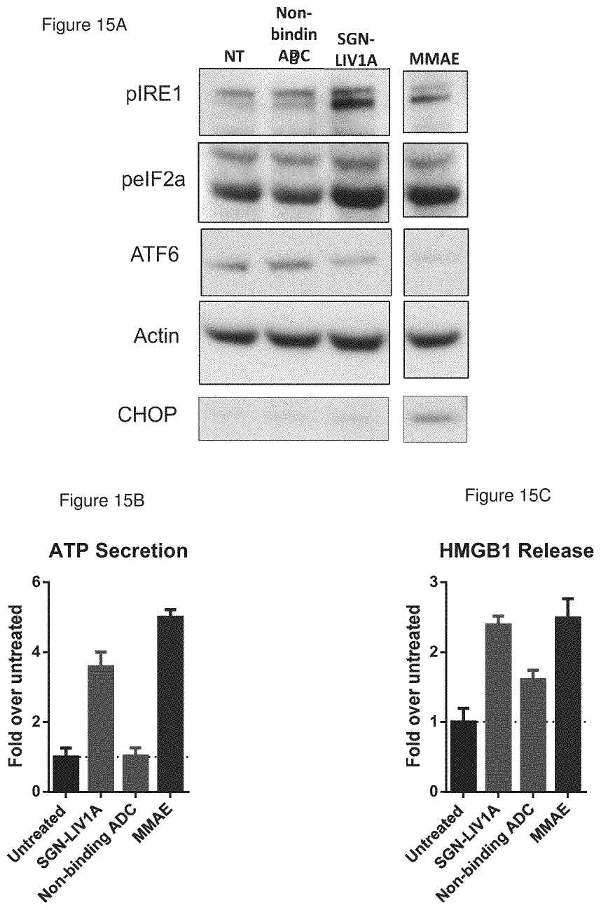

FIGS. 15A-C show the effects of MMAE or an MMAE-containing ADC (Ladiratuzumab vedotin, SGN-LIV1A) on MCF-7 breast cancer cells stress induction (FIG. 15A), ATP secretion (FIG. 15B) and HMGB1 release (FIG. 15C).

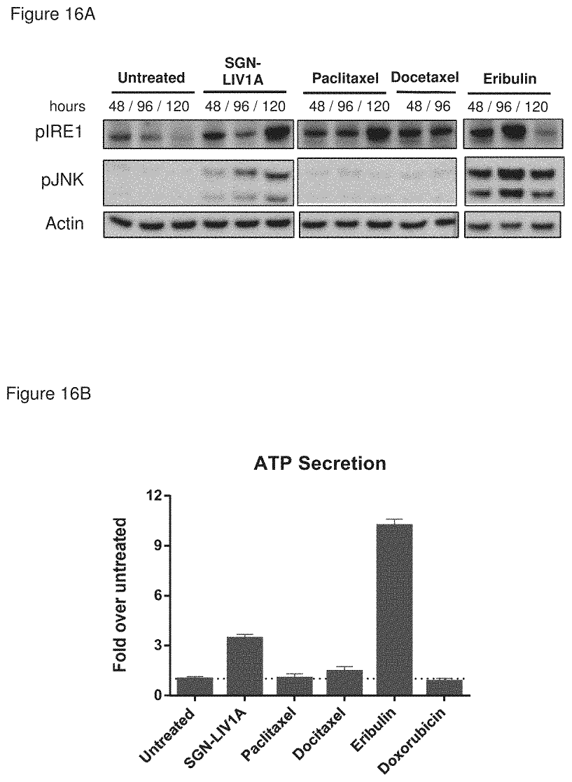

FIGS. 16A-16B show the effects of MMAE, eribulin, paclitaxel, docetaxel or SGN-LIV1A on MCF-7 breast cancer cells stress induction (FIG. 16A) and ATP secretion (FIG. 16B).

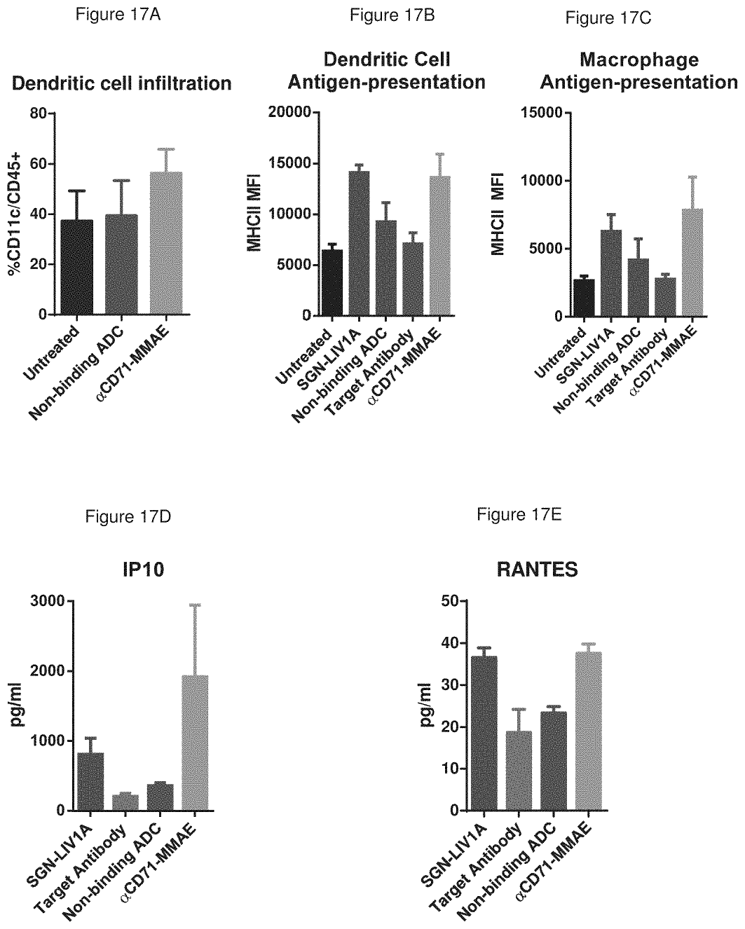

FIGS. 17A-17E shows the effects of MMAE-containing ADC SGN-LIV1A or anti-CD71-MMAE on immune activity in engrafted MCF-7 cells in athymic nude mice: FIG. 17A, dendritic cell infiltration; FIG. 17B, dendritic cell antigen presentation; FIG. 170, macrophage antigen presentation; FIG. 17D, IP10 levels; FIG. 17E, RANTES levels.

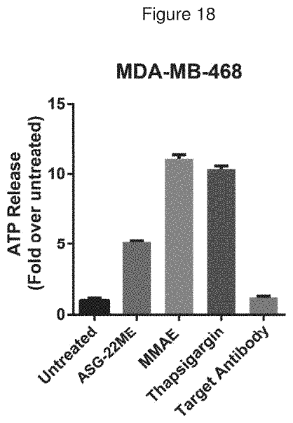

FIG. 18 shows ATP secretion by MDA-MB-468 cells treated with MMAE, thapsigargin or an MMAE-containing ADC (Enfortumab vedotin, ASG-22ME).

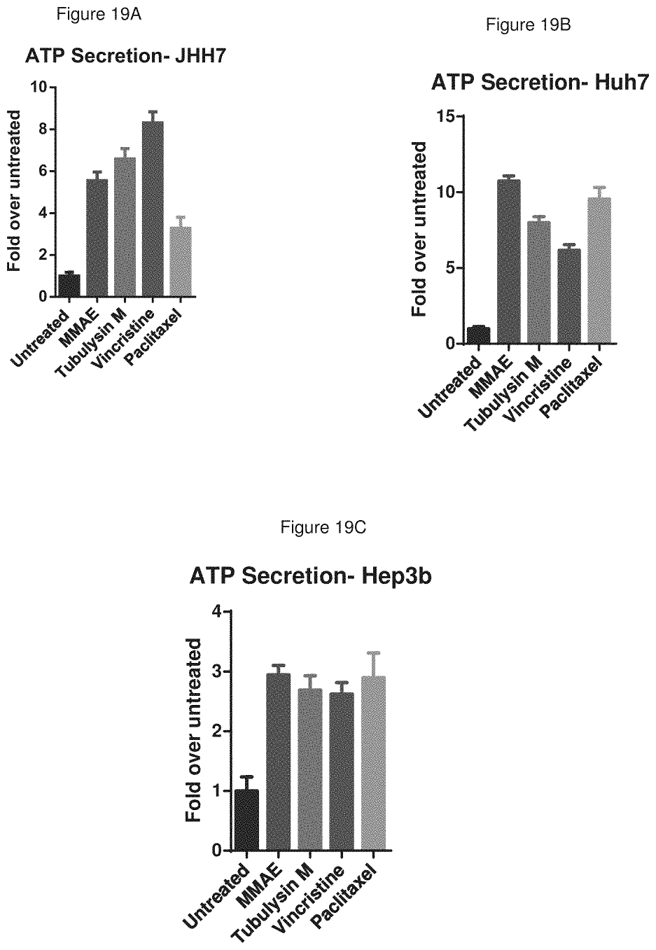

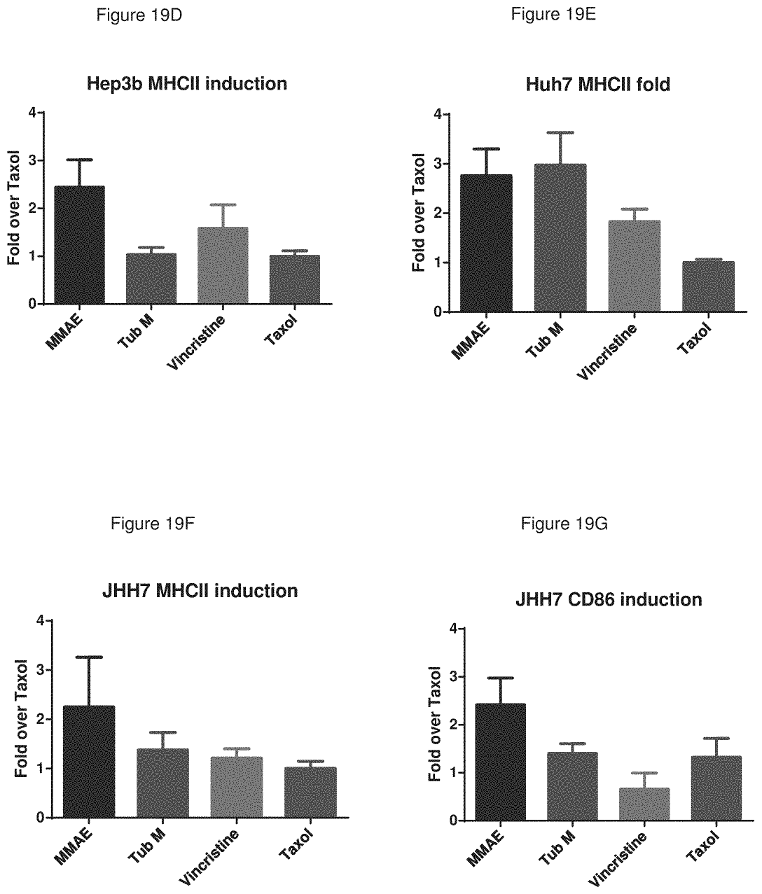

FIGS. 19A-19G show the levels of ATP secretion (FIG. 19A, JHH7; FIG. 19B, Huh7; FIG. 190, Hep3b) and costimulation (as measured by CD86 expression, JHH7) and antigen-presentation (as measured by frequency of MHCII-expressing cells) on Hep3b (FIG. 19D), Huh7 (FIG. 19E), and JHH7 (FIG. 19F-19G) treated with MMAE, Tubulysin M, vincristine, and Paclitaxel.

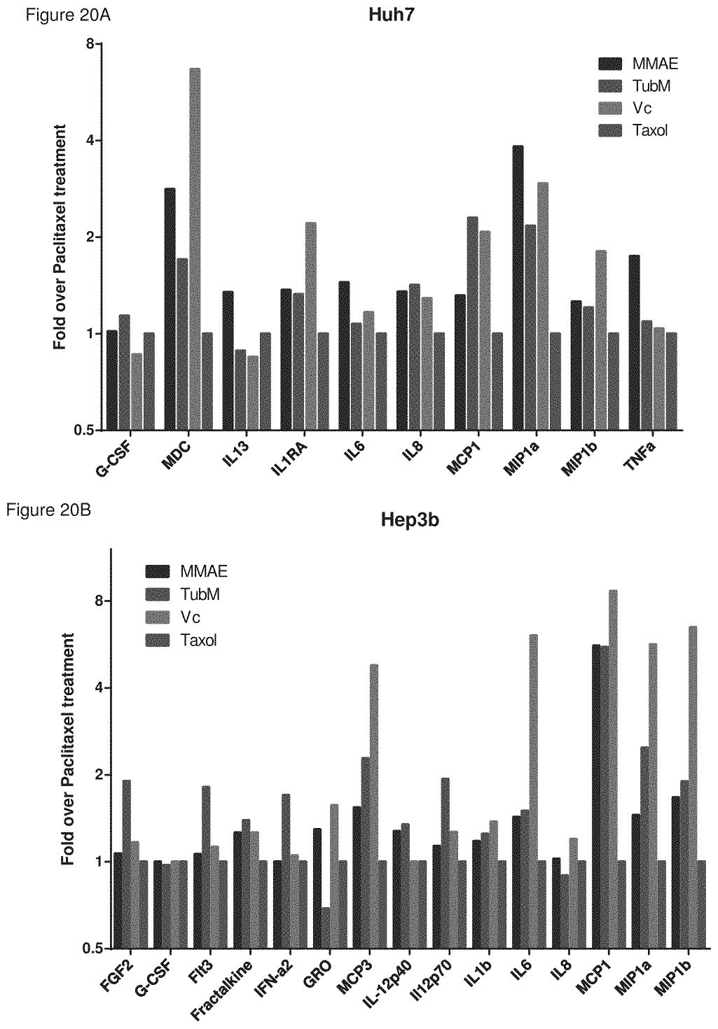

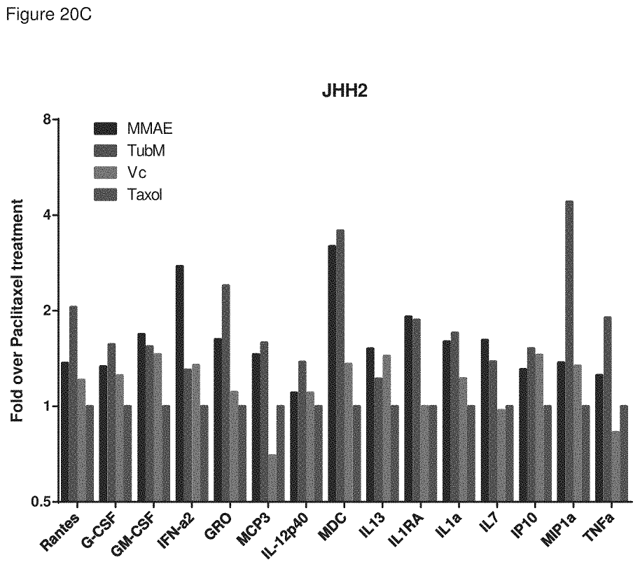

FIGS. 20A-20C show the effects of treatment of Hep3b (FIG. 20A), Huh7 (FIG. 20B), and JHH7 (FIG. 20C) cells with MMAE, Tubulysin M, vincristine or Paclitaxel on levels of cytokines and chemokines.

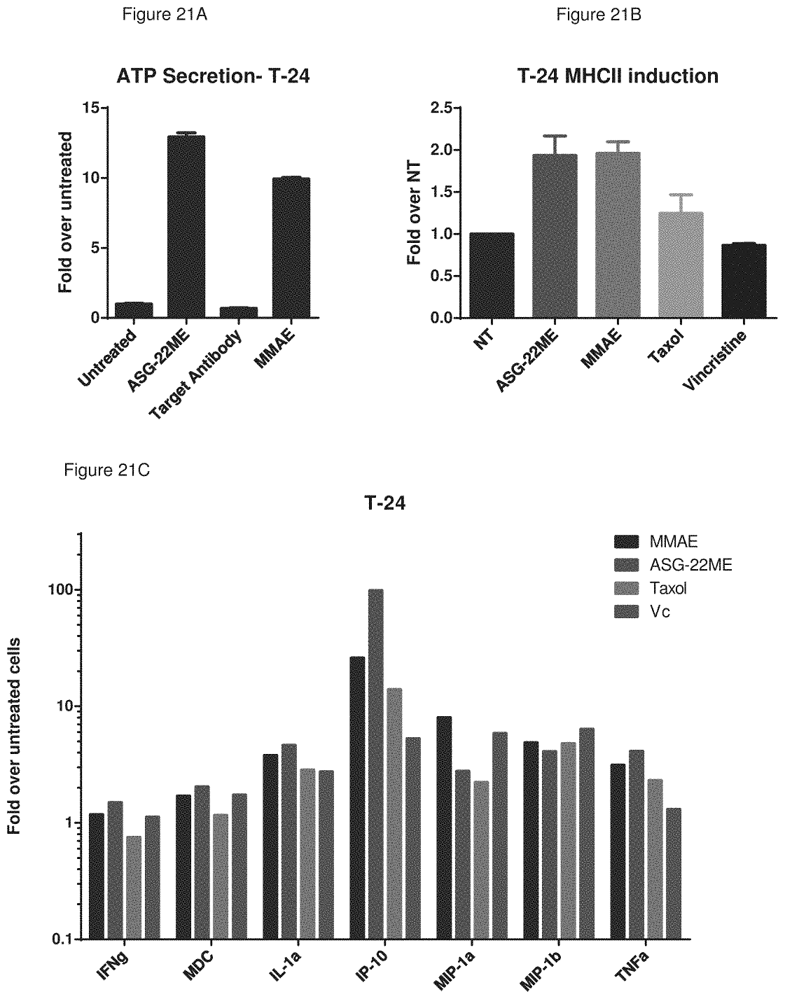

FIGS. 21A-21B show the effects of MMAE, an MMAE-containing ADC (Enfortumab vedotin, ASG-22ME), vincristine, and Paclitaxel on 1-24 bladder tumor cell ATP secretion (FIG. 21A), antigen presentation (FIG. 21B) and cytokine/chemokine production (FIG. 210).

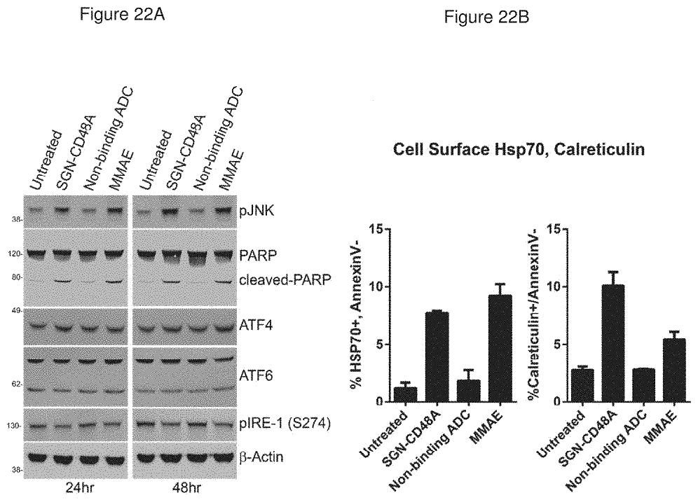

FIGS. 22A-22B shows the effects of MMAE, MMAE-ADC (SGN-CD48A) and control on U-266 cells. FIG. 22A shows Western blot analysis performed using phospho-JNK Thr183/Tyr185 (pJNK), PARP, ATF4, AT6, phospho-IRE-1 Ser274 (pIRE-1); FIG. 22B shows staining for cytotoxic markers HSP70 and calreticulin.

### DETAILED DESCRIPTION

The present disclosure provides methods for treating solid tumors comprising administering an antibody drug conjugate comprising a tubulin disrupting agent. It is disclosed herein that tubulin disrupting agents effect ER stress protein pathways in solid tumor cells and induce ATP secretion and other ER stress phenomena that induce immune cell to migrate to the tumor site and reduce tumor growth.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

As used herein and in the appended claims, the singular forms “a,” “and,” and “the” include plural referents unless the context clearly dictates otherwise. Thus, for example, reference to “a derivative” includes a plurality of such derivatives and reference to “a subject” includes reference to one or more subjects and so forth.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Therapeutically effective amount” or “amount effective to” as used herein refers to that amount of an agent effective to produce the intended beneficial effect on health.

The term “solid tumor” as used herein refers to an abnormal mass of tissue that usually does not contain cysts or liquid areas. Solid tumors may be benign or malignant. Different types of solid tumors are named for the type of cells that form them. Solid tumors include sarcomas and carcinomas. Sarcomas refer to tumors in a blood vessel, bone, fat tissue, ligament, lymph vessel, muscle or tendon. Carcinomas refer to tumors that form in epithelial cells. It is contemplated that the solid tumor is a non-lymphoma solid tumor.

The term “tubulin disrupting agent” refers to an agent that inhibits microtubule function. Tubulin disrupting agents can be classified into two major categories according to their mechanisms of action: agents promoting tubulin polymerization and stabilize microtubule structures and agents that inhibit tubulin polymerization and destabilize microtubule structures. Exemplary tubulin disrupting agents are described in more detail in the Detailed Description.

The term “immune cell migration” as used herein refers to movement of immune cells, including peripheral blood mononuclear cells, T cells, B cells, natural killer cells, monocytes, macrophages, dendritic cells, neutrophils, granulocytes, and the like, to or from a tumor site.

The terms “treat” “treating” or “treatment,” unless otherwise indicated by context, refer to therapeutic treatment and prophylactic measures to prevent progression of or relapse of disease, wherein the object is to inhibit or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder. The term “treating” includes any or all of: inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

Examples of a “patient” or “subject” include, but are not limited to, a human, rat, mouse, guinea pig, monkey, pig, goat, cow, horse, dog, cat, bird and fowl. In an exemplary embodiment, the patient is a human.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which an antibody-drug conjugate is administered.

The terms “specific binding” and “specifically binds” mean that the anti-CD30 antibody will react, in a highly selective manner, with its corresponding target, CD30, and not with the multitude of other antigens.

The term “monoclonal antibody” refers to an antibody that is derived from a single cell clone, including any eukaryotic or prokaryotic cell clone, or a phage clone, and not the method by which it is produced. Thus, the term “monoclonal antibody” as used herein is not limited to antibodies produced through hybridoma technology.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In certain embodiments, the two sequences are the same length.

The term “substantially identical,” in the context of two nucleic acids or polypeptides, refers to two or more sequences or subsequences that have at least 70% or at least 75% identity; more typically at least 80% or at least 85% identity; and even more typically at least 90%, at least 95%, or at least 98% identity (for example, as determined using one of the methods set forth below).

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215; 403-410. BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and PASTA described in Pearson and Lipman, 1988, Proc. Natl, Acad. Sci. 85:2444-8. Alternatively, protein sequence alignment may be carried out using the CLUSTAL W algorithm, as described by Higgins et al., 1996, Methods Enzymol. 266:383-402.

The abbreviation “MMAE” refers to monomethyl auristatin E.

The abbreviations “vc” and “val-cit” refer to the dipeptide valine-citrulline.

The abbreviation “PAB” refers to the self-immolative spacer:

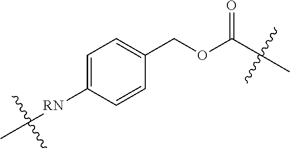

The abbreviation “MC” refers to the stretcher maleimidocaproyl:

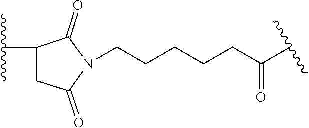 [SMILES: *C(=O)CCCCCN1C(=O)CC(*)C1=O]

cAC10-MC-vc-PAB-MMAE refers to a chimeric AC10 antibody conjugated to the drug MMAE through a MC-vc-PAB linker,

An anti-CD30 vc-PAB-MMAE antibody-drug conjugate refers to an anti-CD30 antibody conjugated to the drug MMAE via a linker comprising the dipeptide valine citrulline and the self-immolative spacer PAB as shown in Formula (I) of U.S. Pat. No. 9,211,319.

### Antibodies

Antibodies of the disclosure are preferably monoclonal, and may be multispecific, human, humanized or chimeric antibodies, single chain antibodies, Fab fragments, F(ab′) fragments, fragments produced by a Fab expression library, and antigen-binding fragments of any of the above. The term “antibody,” as used herein, refers to immunoglobulin molecules and immunologically active portions of immunoglobulin molecules, i.e., molecules that contain an antigen binding site that immunospecifically binds CD30. The immunoglobulin molecules of the disclosure can be of any type (e.g., IgG, IgE, 104, IgD, IgA and IgY), class (e.g., IgG1, IgG2, IgG3, IgG4, IgA1 and IgA2) or subclass of immunoglobulin molecule.

In certain embodiments of the disclosure, the antibodies are human antigen-binding antibody fragments of the present disclosure and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

The antibodies of the present disclosure may be monospecific, bispecific, trispecific or of greater multi specificity. Multispecific antibodies may be specific for different epitopes of CD30 or may be specific for both CD30 as well as for a heterologous protein, See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Antibodies of the present disclosure may be described or specified in terms of the particular CDRs they comprise. The disclosure encompasses an antibody or derivative thereof comprising a heavy or light chain variable domain, said variable domain comprising (a) a set of three CDRs, in which said set of CDRs are from a desired monoclonal antibody, and (b) a set of four framework regions, in which said set of framework regions differs from the set of framework regions in the desired monoclonal antibody, and in which said antibody or derivative thereof immunospecifically binds the target antigen.

In various embodiments, the antibody binds to an antigen on the surface of a cancer cell. In various embodiments, the antibody is specific for CD30, CD19, CD70, CD71, CD20, CD52, CD133, EGFR, HER2, VEGF, VEGFR2, PD-1, PDL1, RANKL, CTLA-4, IL-6, SLAMF7, CD3, TNF-alpha, PDGFR-alpha, CD38, GD2, cCLB8, p97, Nectin-4, or EpCAM.

Anti-CD19 antibodies contemplated for use herein are disclosed, for example, in U.S. Pat. No. 9,073,993. Anti-CD70 antibodies contemplated for use herein are disclosed in, for example, U.S. Pat. No. 9,345,785. Other antibodies that bind cancer-relevant antigens are known in the art, including, but not limited to, rituximab, adalimumab, alemtuzumab, trastuzumab, alemtuzumab, ibritumomab tiuxetan, cetuximab, bevacizumab, panitumumab, ofatumumab, ipilimumab, brentuximab vedotin, pertuzumab, ado-trastuzumab emtansine, obinutuzumab, ramucirumab, pembrolizumab, tositumomab, nivolumab dinutuximab, daratumumab, necitumumab, elotuzumab and atezolizumab.

Murine anti-CD30 mAbs known in the art have been generated by immunization of mice with Hodgkin's disease (HD) cell lines or purified CD30 antigen. AC10, originally termed 010 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16 and Table A below. See also U.S. Pat. No. 7,090,843, incorporated herein by reference, which discloses a chimeric AC10 antibody.

In one aspect, antibodies of the disclosure immunospecifically bind CD30 and exert cytostatic and cytotoxic effects on malignant cells. In certain embodiments antibodies of the disclosure comprise one or more CDRs of AC10.

In a specific embodiment, the disclosure encompasses an anti-CD30 antibody or derivative thereof comprising a heavy chain variable domain, said variable domain comprising (a) a set of three CDRs, in which said set of CDRs comprises SEQ ID NO:4, 6, or 8 and (b) a set of four framework regions, in which said set of framework regions differs from the set of framework regions in monoclonal antibody AC10, and in which said antibody or derivative thereof immunospecifically binds CD30.

In various embodiments, the invention encompasses an antibody or derivative thereof comprising a light chain variable domain, said variable domain comprising (a) a set of three CDRs, in which said set of CDRs comprises SEQ ID NO:12, 14 or 16, and (b) a set of four framework regions, in which said set of framework regions differs from the set of framework regions in monoclonal antibody AC10, and in which said antibody or derivative thereof immunospecifically binds CD30.

Additionally, antibodies of the present disclosure may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions of a known antibody, e.g., AC10, are also included in the present methods. Antibodies of the present disclosure may also be described or specified in terms of their binding affinity to the target antigen. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹M, 10⁻⁹ M, 5×10⁻¹⁰ M, 10⁻¹⁰ 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5)(10⁻¹⁵ M, or 10⁻¹⁵ M.

The antibodies also include derivatives that are modified, by the covalent attachment of any type of molecule to the antibody such that covalent attachment does not prevent the antibody from binding to target antigen. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies contemplated for use in the present invention may be generated by any suitable method known in the art.

The disclosure further provides nucleic acids comprising a nucleotide sequence encoding a protein, including but not limited to, a protein of the disclosure and fragments thereof. Nucleic acids contemplated herein preferably encode one or more CDRs of antibodies that bind to CD30 and exert cytotoxic or cytostatic effects on HD cells. Exemplary nucleic acids of the invention comprise SEQ ID NO:3, SEQ ID NO:5, SEQ ID NO:7, SEQ ID NO:11, SEQ ID NO:13, or SEQ ID NO:15. Variable region nucleic acids of the invention comprise SEQ ID NO:1 or SEQ ID NO:9. (See Table A).

TABLE A

| | NUCLEOTIDE OR | |
|---|---|---|
| MOLECULE | AMINO ACID | SEQ ID NO |
| AC 10 Heavy Chain Variable Region | Nucleotide | 1 |
| AC 10 Heavy Chain Variable Region | Amino Acid | 2 |
| AC 10 Heavy Chain-CDR1 (H1) | Nucleotide | 3 |
| AC 10 Heavy Chain-CDR1 (H1) | Amino Acid | 4 |
| AC 10 Heavy Chain-CDR2 (H2) | Nucleotide | 5 |
| AC 10 Heavy Chain-CDR2 (H2) | Amino Acid | 6 |
| AC 10 Heavy Chain-CDR3 (H3) | Nucleotide | 7 |
| AC 10 Heavy Chain-CDR3 (H3) | Amino Acid | 8 |
| AC 10 Light Chain Variable Region | Nucleotide | 9 |
| AC 10 Light Chain Variable Region | Amino Acid | 10 |
| AC 10 Light Chain-CDR1 (L1) | Nucleotide | 11 |
| AC 10 Light Chain-CDR1 (L1) | Amino Acid | 12 |
| AC 10 Light Chain-CDR2 (L2) | Nucleotide | 13 |
| AC 10 Light Chain-CDR2 (L2) | Amino Acid | 14 |
| AC 10 Light Chain-CDR3 (L3) | Nucleotide | 15 |
| AC 10 Light Chain-CDR3 (L3) | Amino Acid | 16 |

In various embodiments, the antibody is an IgG antibody, e.g. an IgG1, IgG2, IgG3 or IgG4 antibody, preferably an IgG1 antibody.

### Antibody-Drug Conjugates

Contemplated herein is the use of Drug-Linker Unit-antibody conjugates, or antibody drug conjugates, comprising tubulin disrupting agents to treat solid tumors.

Several different categories of tubulin disrupting agent are known in the field, including, auristatins, tubulysins, colchicine, vinca alkaloids, taxanes, cryptophycins, maytansinoids, hemiasterlins, and other tubulin disrupting agents.

Auristatins are derivatives of the natural product dolastatin. Exemplary auristatins include dolostatin-10, MMAE (N-methylvaline-valine-dolaisoleuine-dolaproine-norephedrine) and MMAF (N-methylvaline-valine-dolaisoleuine-dolaproine-phenylalanine) and AFP. WO 2015/057699 describes PEGylated auristatins including MMAE. Additional dolostatin derivatives contemplated for use are disclosed in U.S. Pat. No. 9,345,785, incorporated herein by reference.

Tubulysins include, but are not limited to, tubulysin D, tubulysin M, tubuphenylalanine and tubutyrosine. WO 2017-096311 and WO 2016-040684 describe tubulysin analogs including tubulysin M.

Colchicines include, but are not limited to, colchicine and CA-4.

Vinca alkaloids include, but are not limited to, Vinblastine (VBL), vinorelbine (VRL), vincristine (VCR) and vindesine (VDS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs, DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions. Maytansinoid drug moieties also include those having modifications such as: C-9-SH (U.S. Pat. No. 4,424,219) (prepared by the reaction of maytansinol with H.sub.25 or P.sub.25.sub.5); C-14-alkoxymethyl(demethoxy/CH.sub.2OR) (U.S. Pat. No. 4,331,598); C-14-hydroxymethyl or acyloxymethyl (CH.sub.2OH or CH.sub.2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol). The cytotoxicity of the TA.1-maytansonoid conjugate that binds HER-2 (Chari et al., Cancer Research 52:127-131 (1992) was tested in vitro on the human breast cancer cell line SK-BR-3. The drug conjugate achieved a degree of cytotoxicity similar to the free maytansinoid drug, which could be increased by increasing the number of maytansinoid molecules per antibody molecule.

Hemiasterlins include but are not limited to, hemiasterlin and HT1-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

The Drug-Linker Unit-Antibody, or antibody drug conjugates, contemplated for use in the methods herein comprise linker units Typically, the ADC or ADC derivative comprises a linker region between the therapeutic agent and the antibody or derivative thereof. The linker may be a protease cleavable linker, an acid-cleavable linker, a disulfide linker a self-stabilizing linker. In various embodiments, the linker is cleavable under intracellular conditions, such that cleavage of the linker releases the therapeutic agent from the antibody in the intracellular environment.

For example, in some embodiments, the linker is cleavable by a cleaving agent that is present in the intracellular environment (e.g., within a lysosome or endosome or caveolae). The linker can be, e.g., a peptidyl linker that is cleaved by an intracellular peptidase or protease enzyme, including, but not limited to, a lysosomal or endosomal protease. Typically, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, e.g., in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high. See also U.S. Pat. No. 9,345,785.

The terms “intracellularly cleaved” and “intracellular cleavage” refer to a metabolic process or reaction inside a cell on an antibody drug conjugate, whereby the covalent attachment, e.g., the Linker, between the Drug moiety (D) and the Antibody unit is broken, resulting in the free Drug, or other metabolite of the conjugate dissociated from the antibody inside the cell. The cleaved moieties of the Drug-Linker Unit-Ab conjugate are thus intracellular metabolites.

In various embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharma Therapeutics 83:67-123; Neville et at, 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

In various embodiments, the linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In various embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In some embodiments, the linker unit is not cleavable and the drug is released by antibody degradation. (See U.S. Publication No. 2005/0238649).

In various embodiments, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of ADC or ADC derivative, are cleaved when the ADC or ADC derivative present in an extracellular environment (e.g., in plasma). Whether a linker is not substantially sensitive to the extracellular environment can be determined, for example, by incubating independently with plasma both (a) the ADC or ADC derivative (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

In various embodiments, the linker promotes cellular internalization. In certain embodiments, the linker promotes cellular internalization when conjugated to the therapeutic agent (i.e., in the milieu of the linker-therapeutic agent moiety of the ADC or ADC derivate as described herein). In yet other embodiments, the linker promotes cellular internalization when conjugated to both the drug and the antigen-specific antibody or derivative thereof (i.e., in the milieu of the ADC or ADC derivative as described herein).

A variety of linkers that can be used with the present compositions and methods are described in WO 2004010957 entitled “Drug Conjugates and Their Use for Treating Cancer, An Autoimmune Disease or an Infectious Disease” filed Jul. 31, 2003.

In various embodiments, the protease cleavable linker comprises a thiolreactive spacer and a dipeptide. In some embodiments, the protease cleavable linker consists of a thiolreactive maleimidocaproyl spacer, a valine-citrulline dipeptide, and a p-amino-benzyloxycarbonyl spacer.

In various embodiments, the acid cleavable linker is a hydrazine linker or a quaternary ammonium linker.

Self-stabilizing linkers comprising a maleimide group are described in U.S. Pat. No. 9,504,756, herein incorporated by reference.

In various embodiments, the tubulin disrupting agent, such as auristatin, is conjugated to a linker by a C-terminal carboxyl group that forms an amide bond with the Linker Unit as described in U.S. Pat. No. 9,463,252, incorporated herein by reference. In various embodiments, the Linker unit comprises at least one amino acid. Binder-drug conjugates (ADCs) of N,N-dialkylauristatins are disclosed in U.S. Pat. No. 8,992,932

In various embodiments, the linker also comprises a stretcher unit and/or an amino acid unit. Exemplary stretcher units and amino acid units are described in U.S. Pat. Nos. 9,345,785 and 9,078,931, each of which is herein incorporated by reference.

In various embodiments, provided herein is the use of antibody drug conjugates comprising an anti-CD30 antibody, covalently linked to MMAE through a vc-PAB linker. The antibody drug conjugates are delivered to the subject as a pharmaceutical composition. CD30 antibody drug conjugates are described in U.S. Pat. No. 9,211,319, herein incorporated by reference.

In various embodiments, the Drug-Linker Unit-Antibody/antibody-drug conjugates of the present invention have the following formula:

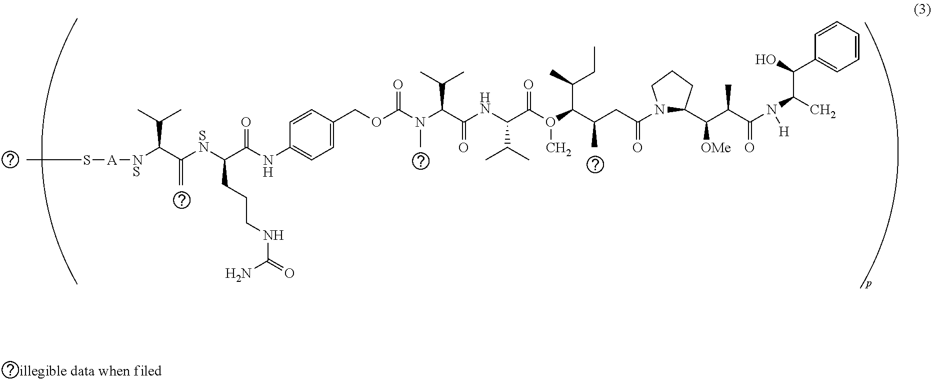

or a pharmaceutically acceptable salt thereof; wherein: mAb is an monoclonal antibody, such as anti-CD30 or anti-CD19 antibody, S is a sulfur atom of the antibody A− is a Stretcher unit, p is from about 3 to about 5.

The drug loading is represented by p, the average number of drug molecules per antibody in a pharmaceutical composition. For example, if p is about 4, the average drug loading taking into account all of the antibody present in the pharmaceutical composition is about 4. P ranges from about 3 to about 5, more preferably from about 3.6 to about 4.4, even more preferably from about 3.8 to about 4.2. P can be about 3, about 4, or about 5. The average number of drugs per antibody in preparation of conjugation reactions may be characterized by conventional means such as mass spectroscopy, ELISA assay, and HPLC. The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

The Stretcher unit (A), is capable of linking an antibody unit to the valine-citrulline amino acid unit via a sulfhydryl group of the antibody. Sulfhydryl groups can be generated, for example, by reduction of the interchain disulfide bonds of an antigen-specific antibody. For example, the Stretcher unit can be linked to the antibody via the sulfur atoms generated from reduction of the interchain disulfide bonds of the antibody. In some embodiments, the Stretcher units are linked to the antibody solely via the sulfur atoms generated from reduction of the interchain disulfide bonds of the antibody. In some embodiments, sulfhydryl groups can be generated by reaction of an amino group of a lysine moiety of an antibody with 2-iminothiolane (Traut's reagent) or other sulfhydryl generating reagents. In certain embodiments, the antibody is a recombinant antibody and is engineered to carry one or more lysines. In certain other embodiments, the recombinant antibody is engineered to carry additional sulfhydryl groups, e.g., additional cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945 each of which is incorporated herein by reference in its entirety.

Representative Stretcher units are described within the square brackets of Formulas IIIa and IIIb of U.S. Pat. No. 9,211,319, and incorporated herein by reference.

In various embodiments, the antibody drug conjugate comprises monomethyl auristatin E and a protease-cleavable linker. It is contemplated that the protease cleavable linker is comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In a preferred embodiment, the antibody drug conjugate is brentuximab vedotin, an antibody-drug conjugate which has the structure:

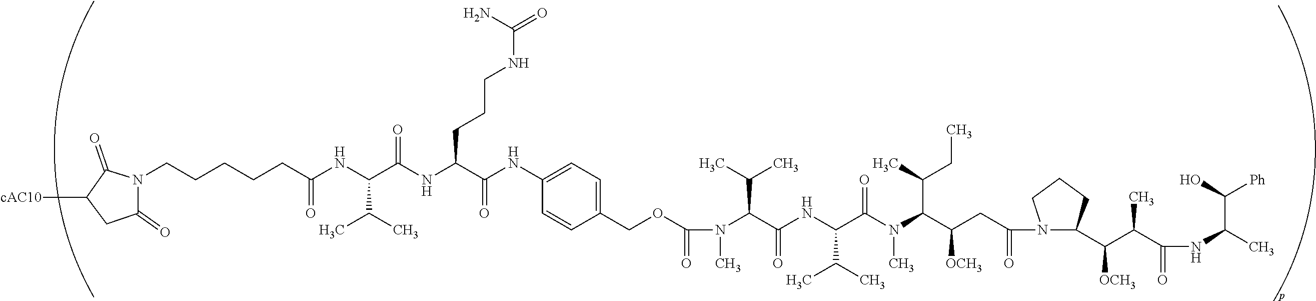

Brentuximab vedotin is a CD30-directed antibody-drug conjugate consisting of three components: (i) the chimeric IgG1 antibody cAC10, specific for human CD30, (ii) the microtubule disrupting agent MMAE, and (in) a protease—cleavable linker that covalently attaches MMAE to cAC10. The drug to antibody ratio or drug loading is represented by “p” in the structure of brentuximab vedotin and ranges in integer values from 1 to 8. The average drug loading brentuximab vedotin in a pharmaceutical composition is about 4.

### Methods of Use

Provided herein are methods for treating a solid tumor comprising administering an antibody-drug conjugate comprising a tubulin disrupting agent to treat a solid tumor. In various embodiments, it is contemplated that the methods of the present disclosure treat solid tumors by inducing ER stress pathways after disruption of mictrotubule function. In various embodiments, the antibody drug conjugate agent comprising a tubulin disrupting agent induces apoptosis in the solid tumor.

In various embodiments, the antibody drug conjugate agent comprising a tubulin disrupting agent increases immune cell migration to a solid tumor.

It is demonstrated in the Examples, that tubulin-disrupting agents increase ER stress protein pathways, such as increasing ATP secretion and increasing High mobility group box 1 (HMGB1) protein levels, which results in increased apoptosis of cells, which can, in turn, draw immune cells to the site of apoptosis and cell stress.

It is contemplated that the methods herein reduce tumor size or tumor burden in the subject, and/or reduce metastasis in the subject. In various embodiments, tumor size in the subject is decreased by about 25-50%, about 4070% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

It is contemplated that the methods herein reduce tumor burden, and also reduce or prevent the recurrence of tumors once the cancer has gone into remission

Exemplary solid tumors contemplated herein include lung cancer, breast cancer, ovarian cancer, cervical cancer, gastrointestinal cancers, head and neck cancer, melanoma, sarcoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm, renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage WA skin melanoma; stage IIIB skin melanoma, stage IIIC skin melanoma; stage IV skin melanoma, malignant melanoma of head and neck, lung cancer, non small cell lung cancer (NSCLC), squamous cell non-small cell king cancer, breast cancer, recurrent metastatic breast cancer, hepatocellular carcinoma, richter's syndrome; waldenstrom macroglobulinemia, adult glioblastoma; adult gliosarcoma, recurrent glioblastoma, recurrent childhood rhabdomyosarcoma, recurrent ewing sarcoma/peripheral primitive neuroectodermal tumor, recurrent neuroblastoma; recurrent osteosarcoma, colorectal cancer, MS positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage HI merkel cell carcinoma; stage IV merkel cell carcinoma, myelodysplastic syndrome and Sezary syndrome. In one embodiment, the solid tumor is a non-lymphoma solid tumor. In some embodiments, the solid tumor may be multiple myeloma.

In various embodiments, the ADC may be administered with one or more chemotherapeutics. Exemplary chemotherapeutic agents are disclosed in the following table and may be used alone or in combination with one or more additional chemotherapeutic agents, which in turn can also be administered in combination with an antibody drug conjugate.

Chemotherapeutic Agents

| | |
|---|---|
| Alkylating agents | Natural products |
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vindesine |
| carmustine (BCNU) | vinorelbin |
| lomustine (CCNU) | Taxotere ® (docetaxel) |
| semustine (methyl-CCNU) | estramustine |
| Ethylenimine/Methyl-melamine | estramustine phosphate |
| thriethylenemelamine (TEM) | Epipodophylotoxins |
| triethylene thiophosphoramide | etoposide |
| (thiotepa) | teniposide |
| hexamethylmelamine | Antibiotics |
| (HMM, altretamine) | actimomycin D |
| Alkyl sulfonates | daunomycin (rubido-mycin) |
| busulfan | doxorubicin (adria-mycin) |
| Triazines | mitoxantrone |
| dacarbazine (DTIC) | idarubicin |
| Antimetabolites | epirubicin |
| Folic Acid analogs | valrubicin |
| methotrexate | bleomycin |
| Trimetrexate | splicamycin (mithramycin) |
| Pemetrexed | mitomycinC |
| (Multi-targeted antifolate) | dactinomycin |
| Pyrimidine analogs | aphidicolin |
| 5-fluorouracil | Enzymes |
| fluorodeoxyuridine | L-asparaginase |
| gemcitabine | L-arginase |
| cytosine arabinoside | Radiosensitizers |
| (AraC, cytarabine) | metronidazole |
| 5-azacytidine | misonidazole |
| 2,2′- difluorodeoxy-cytidine | desmethylmisonidazole |
| Purine analogs | pimonidazole |
| 6-mercaptopurine | etanidazole |
| 6-thioguanine | nimorazole |
| azathioprine | RSU 1069 |
| 2′-deoxycoformycin | EO9 |
| (pentostatin) | RB 6145 |
| erythrohydroxynonyl-adenine | SR4233 |
| (EHNA) | |
| fludarabine phosphate | nicotinamide |
| 2-chlorodeoxyadenosine | 5-bromodeozyuridine |
| (cladribine, 2-CdA) | 5-iododeoxyuridine |
| Type 1 Topoisomerase Inhibitors | bromodeoxycytidine |
| camptothecin | Miscellaneous agents |
| topotecan | bisphosphonates |
| irinotecan | RANKL inhibitor |
| Biological response modifiers | denosumab |
| G-CSF | Platinium coordination complexes |
| GM-CSF | cisplatin |
| Differentiation Agents | carboplatin |
| retinoic acid derivatives | oxaliplatin |
| Hormones and antagonists | nthracenedione |
| Adrenocorticosteroids/antagonists | mitoxantrone |
| calcitonin | Substituted urea |
| prednisone and equiv-alents | hydroxyurea |
| dexamethasone | Methylhydrazine derivatives |
| ainoglutethimide | N-methylhydrazine (MIH) |
| Progestins | procarbazine |
| hydroxyprogesterone caproate | Adrenocortical suppressant |
| medroxyprogesterone acetate | mitotane (o, p′-DDD) |
| megestrol acetate | ainoglutethimide |
| Estrogens | Cytokines |
| diethylstilbestrol | interferon (α, β, γ) |
| ethynyl estradiol/equivalents | interleukin-2 |
| Antiestroqen | Photosensitizers |
| tamoxifen | hematoporphyrin derivatives |
| Androgens | Photofrin ® |
| testosterone propionate | benzoporphyrin derivatives |
| fluoxymesterone/equivalents | Npe6 |
| Antiandroqens | tin etioporphyrin (SnET2) |
| flutamide | pheoboride-a |
| gonadotropin-releasing | bacteriochlorophyll-a |
| hormone analogs | naphthalocyanines |
| leuprolide | phthalocyanines |
| Nonsteroidal antiandroqens | zinc phthalocyanines |
| flutamide | Radiation |
| Histone Deacetylase Inhibitors | X-ray |
| Vorinostat | ultraviolet light |
| Romidepsin | gamma radiation |
| | visible light |
| | infrared radiation |
| | microwave radiation |

In various embodiments, therapy is administered on a period basis, for example, twice weekly, weekly, every 2 weeks, every 3 weeks, monthly, once every two months or at a longer interval. In a related embodiment, in exemplary treatments, an antibody drug conjugate is administered in a dose range of 0.1 to 15 mg/kg.

In one aspect, methods of the present disclosure include a step of administering a pharmaceutical composition. In certain embodiments, the pharmaceutical composition is a sterile composition.

Methods of the present disclosure are performed using any medically-accepted means for introducing therapeutics directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the disclosure implanted near or at site of the cancer.

Therapeutic compositions may also be delivered to the patient at multiple sites. The multiple administrations may be rendered simultaneously or may be administered over a period of time. In certain cases it is beneficial to provide a continuous flow of the therapeutic composition.

Also contemplated in the present disclosure is the administration of multiple agents, such as the antibody compositions in conjunction with another agent as described herein, including but not limited to a chemotherapeutic agent.

The amounts of antibody drug conjugate composition in a given dosage may vary according to the size of the individual to whom the therapy is being administered as well as the characteristics of the disorder being treated. In exemplary treatments, it may be necessary to administer about 1 mg/day, 5 mg/day, 10 mg/day, 20 mg/day, 50 mg/day, 75 mg/day, 100 mg/day, 150 mg/day, 200 mg/day, 250 mg/day, 500 mg/day or 1000 mg/day. These concentrations may be administered as a single dosage form or as multiple doses. Standard dose-response studies, first in animal models and then in clinical testing, reveals optimal dosages for particular disease states and patient populations.

Also contemplated is a composition comprising any of the foregoing antibody drug conjugates, or use thereof in preparation of a medicament, for treatment of a solid tumor. Syringes, e.g., single use or pre-filled syringes, sterile sealed containers, e.g. vials, bottle, vessel, and/or kits or packages comprising any of the foregoing antibodies or compositions, optionally with suitable instructions for use, are also contemplated.

### Formulations

Various delivery systems can be used to administer the Drug-Linker Unit-Antibody conjugate/antibody-drug conjugates. In certain embodiments of the disclosure, administration of the antibody-drug conjugate compound is by intravenous infusion or by subcutaneous injection. In some embodiments, administration is by a 30 minute, 1 hour or two hour intravenous infusion.

The antibody-drug conjugate compound can be administered as a pharmaceutical composition comprising one or more pharmaceutically compatible ingredients. For example, the pharmaceutical composition typically includes one or more pharmaceutically acceptable carriers, for example, water-based carriers (e.g., sterile liquids). Water is a more typical carrier when the pharmaceutical composition is administered intravenously.

The composition, if desired, can also contain, for example, saline salts, buffers, salts, nonionic detergents, and/or sugars. Examples of suitable pharmaceutical carriers are described in “Remington's Pharmaceutical Sciences” by E. W. Martin. The formulations correspond to the mode of administration.

The present disclosure provides, for example, pharmaceutical compositions comprising a therapeutically effective amount of the antibody-drug conjugate, a buffering agent, optionally a cryoprotectant, optionally a bulking agent, optionally a salt, and optionally a surfactant. Additional agents can be added to the composition. A single agent can serve multiple functions. For example, a sugar, such as trehalose, can act as both a cryoprotectant and a bulking agent. Any suitable pharmaceutically acceptable buffering agents, surfactants, cyroprotectants and bulking agents can be used in accordance with the present invention.

In addition to providing methods for treating a hematological cancer, the present invention provides antibody drug conjugate formulations including drug conjugate formulations that have undergone lyophilization, or other methods of protein preservation, as well as antibody drug formulations that have not undergone lyophilization.

In some embodiments, the antibody drug conjugate formulation comprises (i) about 1-25 mg/ml, about 3 to about 10 mg/ml of an antibody-drug conjugate, or about 5 mg/ml (e.g., an antibody-drug conjugate of formula I or a pharmaceutically acceptable salt thereof), (ii) about 5-50 mM, preferably about 10 mM to about 25 mM of a buffer selected from a citrate, phosphate, or histidine buffer or combinations thereof, preferably sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

In some embodiments, an antibody drug conjugate formulation will comprise about 1-25 mg/ml, about 3 to about 10 mg/nil, preferably about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose or sucrose or combinations thereof, optionally (iv) about 0.05 to about 1 mg/ml of a surfactant selected from polysorbate 20 or polysorbate 80, and (v) water, wherein the pH of the composition is from about 5.3 to about 7, preferably about 6.6.

In some embodiments, an antibody drug conjugate formulation will comprise about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose, optionally (iv) about 0.05 to about 1 mg/ml of a surfactant selected from polysorbate 20 or polysorbate 80, and (v) water, wherein the pH of the composition is from about 5.3 to about 7, preferably about 6.6.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present invention can be used with the methods described herein or with other methods for treating disease. The antibody drug conjugate formulations may be further diluted before administration to a subject. In some embodiments, the formulations will be diluted with saline and held in IV bags or syringes before administration to a subject. Accordingly, in some embodiments, the methods for treating a hematologic cancer in a subject will comprise administering to a subject in need thereof a weekly dose of a pharmaceutical composition comprising antibody-drug conjugates having formula I wherein the administered dose of antibody-drug conjugates is from about 1.8 mg/kg or 1.2 mg/kg of the subject's body weight to 0.9 mg/kg of the subject's body weight and the pharmaceutical composition is administered for at least three weeks and wherein the antibody drug conjugates, prior to administration to a subject, were present in a formulation comprising (i) about 1-25 mg/ml, preferably about 3 to about 10 mg/ml of the antibody-drug conjugate (ii) about 5-50 mM, preferably about 10 mM to about 25 mM of a buffer selected from sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

Formulations of chemotherapeutics may be contemplated for use herein, including doxorubicin, vinblastine, dacarbazine, cyclophosphamide, vincristine, or prednisone are provided as typically used in the treatment of cancers. For example, doxorubicin, vinblastine, dacarbazine cyclophosphamide, vincristine, and prednisone are commercially available and approved by the United States FDA and other regulatory agencies for use in treating patients with multiple types of cancer.

The present invention also provides kits for the treatment of a solid tumor. The kit can comprise (a) a container containing the antibody-drug conjugate and optionally, containers comprising one or more chemotherapeutic. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### EXAMPLES

### Example 1- Effects of Tubulin Disrupting Agents on Solid Tumor

The effects of tubulin disrupting agents on solid tumor cell lines were assessed. Cancer cells were treated with MMAE and assessed for the following immunogenic cell death (ICD) characteristics; ER stress, AIR secretion and extracellular HMGB1 levels.

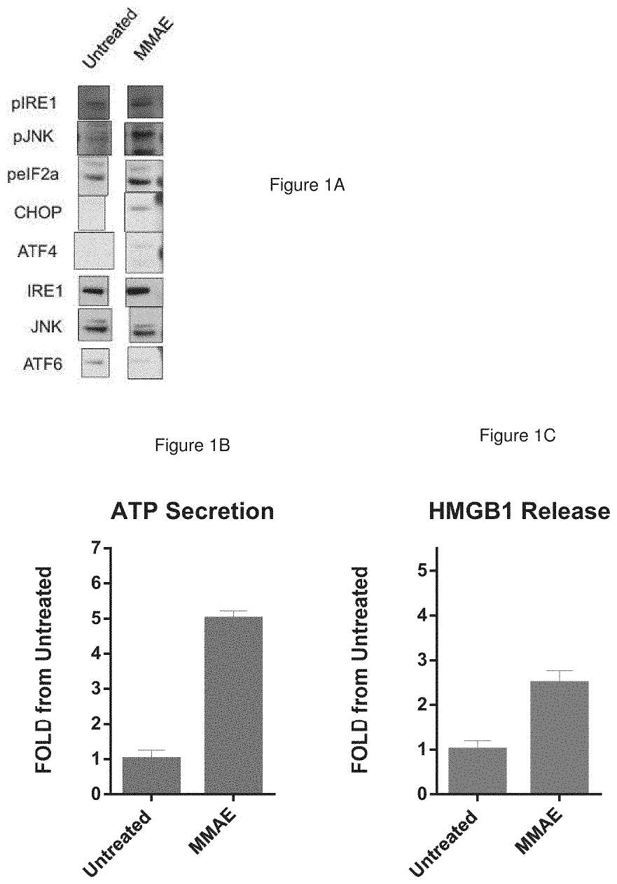

MCF7 breast cancer cells were treated with 100 nM MMAE for 16 hours and harvested RIPA buffer for western blot analysis. Treatment with MMAE activated all 3 pathways of the ER stress response, as indicated by phosphorylation of IRE1 and eIF2a (FIG. 1A), as well as cleavage of full-length ATF6. Severe ER stress is a prerequisite to the exposure of pro-phagocytic signals on the surface of tumor cells, and is elicited by MMAE as indicated by activation of MK signaling by phosphorylated IRE1, and expression of CHOP.

Induction of ICD is also characterized by the secretion of ATP and HMGB1. Extracellular ATP serves as a strong chemotactic signal, promoting immune cell migration to the tumor site. Upon arrival, extracellular HMGB1 signals through various pro-inflammatory receptors (TLR2, TLR4, RAGE) to activate antigen-presenting cells, thereby promoting immune activity within the tumor. Treatment of MCF7 cells with MMAE leads to increased secretion of ATP and HMGB1 (FIGS. 1B, 1C).

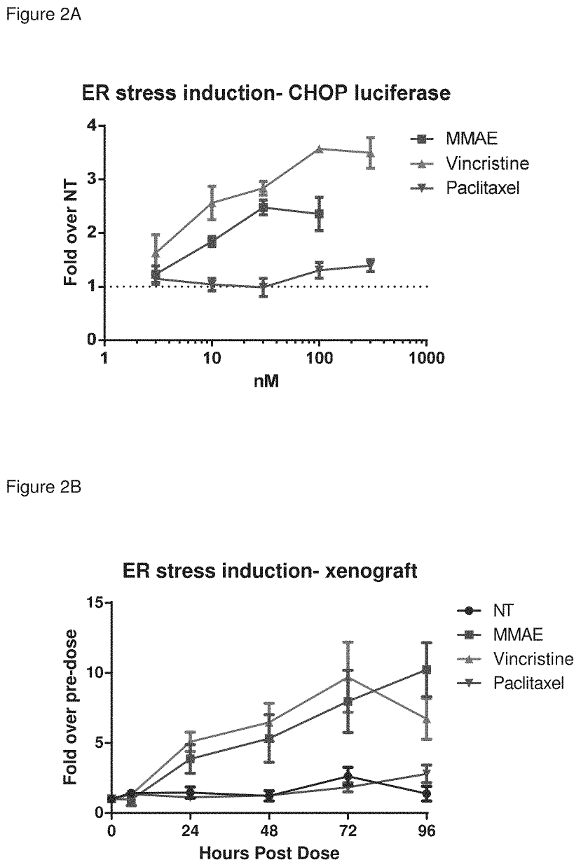

Severe ER stress leads to upregulation of CHOP, and initiates mitochondrial apoptosis. MiaPaca2 pancreatic tumor cells were engineered to express a CHOP-driven luciferase reporter system (purchased from Signosis, Inc.) that allows for quantifiable monitoring of severe ER stress. MiaPaca-CHOP-luciferase cells were treated with MMAE, vincristine, and Paclitaxel, and assayed for luciferase expression after 16 hours. Treatment with MMAE and vincristine exhibited dose-dependent increase in luciferase signal as a proxy of severe ER stress induction, while Paclitaxel induced a modest luciferase signal at the peak doses FIG. 2A).

MiaPaca-CHOP-luciferase cells were subcutaneously engrafted into NOD/SCID/gamma-chain deficient mice. Subcutaneous tumors were treated intratumorally with MMAE (0.36 mg/kg), vincristine (1.0 mg/kg), and Paclitaxel (10 mg/kg) and tumoral luciferase signal was monitored over time. As evidenced, treatment with MMAE and vincristine rapidly elicited severe ER stress in engrafted tumors, whereas Paclitaxel does not induce ER stress (FIG. 2B).

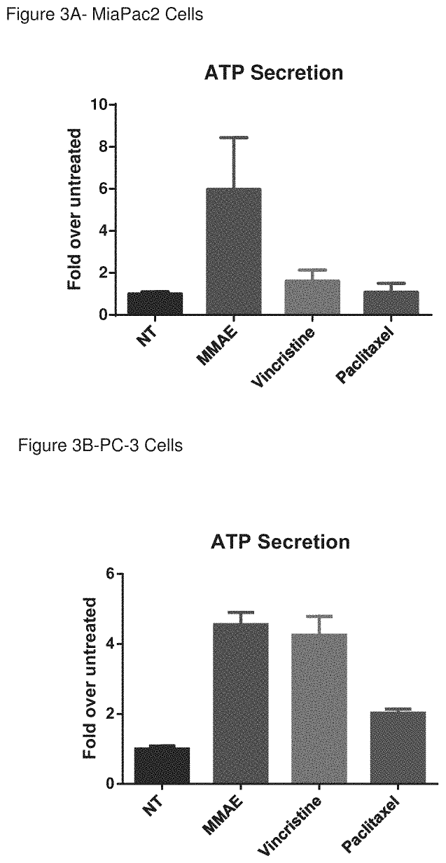

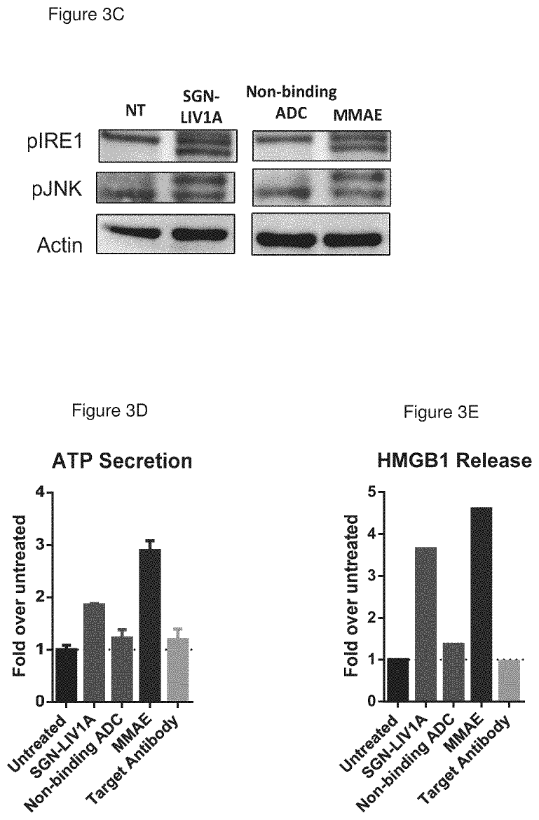

MiaPaca2 cells were treated with MMAE, Vincristine, or Paclitaxel. Supernatant was collected after 16 hours of treatment, and analyzed for ATP secretion. Treatment with MMAE elicited robust ATP secretion, whereas Paclitaxel treatment resulted in modest ATP secretion (FIG. 3A).

PC-3 prostate tumor cells were also treated with MMAE, Vincristine, or Paclitaxel. Supernatant was collected after 16 hours of treatment, and analyzed for ATP secretion. Treatment with MMAE and vincristine elicited robust ATP secretion, whereas Paclitaxel treatment resulted in modest ATP secretion (FIG. 3B). PC-3 cells were treated for 24 hours with an MMAE-containing ADC (SGN-LIV1A) or MMAE and harvested in RIPA buffer for western blot analysis (FIG. 3C). Treatment with MMAE elicits ER stress (phosphorylation of IRE1 and JNK) and release of ATP and HMGB1, resulting in the induction of immunogenic cell death (FIG. 3D-3E).

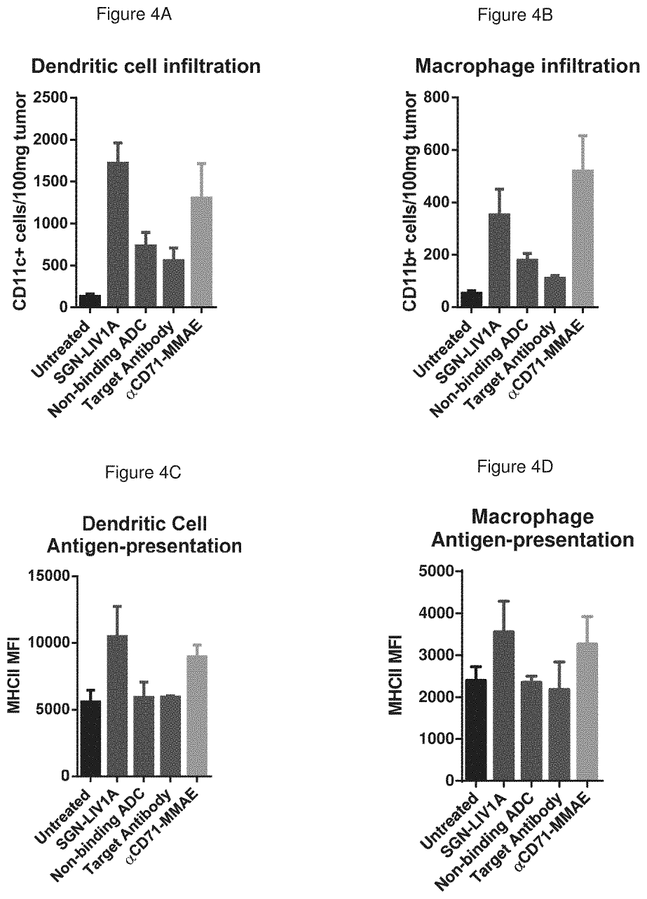

Athymic nude mice were subcutaneously engrafted with PC-3 cells. Upon reaching 200 cubic millimeters, mice received a single intraperitoneal dose of an MMAE-containing ADC (SGN-LIV1A or anti-CD71-MMAE). 8 days post-ADC treatment, tumors were excised and assessed for immune cell infiltration and composition by flow cytometry. Tumors treated with MMAE-ADCs exhibited increased infiltration of immune cells which further showed enhanced activation (FIGS. 4A-D).

In another study, athymic nude mice were subcutaneously engrafted with PC-3 cells. Upon reaching 200 cubic millimeters, mice received a single intraperitoneal dose of an MMAE-containing ADC (SGN-LIV1A or anti-CD71-MMAE). 8 days post-ADC treatment, tumors were excised and homogenized in RIPA buffer and cytokine/chemokine production was measured by ELISA. Peripheral cytokine levels were also measured in the serum by ELISA. In addition to increased immune cells within the tumor, there is enhanced immune activity as evidenced by elevated cytokine and chemokine production from these immune cells (FIG. 5A-F).

HeLa cervical cancer cells were treated with 1000 nM or 100 nM MMAE, Vincristine, or Paclitaxel for 16 hours and harvested for western blot analysis. Each treatment activated ER stress responses, as indicated by phosphorylation of IRE1. However, MMAE elicited a more severe ER stress response that was sustained with decreasing doses, as evidenced by further phosphorylation of JNK. Robust JNK phosphorylation was not seen with lower doses of Paclitaxel (FIG. 6).

Skin tumor lines A2058, SK-MEL-5 and SK-MEL-28, Calu-1 (lung), HT-1080 (fibrosarcoma), SK-MES-1 (lung), and BXPC3 (pancreas) cells were treated with MMAE, vincristine, and Paclitaxel. Supernatant was collected after 17 hours of treatment, and analyzed for ATP secretion. Treatment with MMAE and vincristine elicited ATP secretion in most of the cell lines assayed (6/7), and was able to induce a more robust response than Paclitaxel in all cell lines assayed (FIG. 7A). Treatment with MMAE elicited potent ATP secretion from all 3 A2058, SK-MEL-5, SK-MEL-28 (skin) cell lines, whereas Paclitaxel elicited ATP secretion from only 1 of 3 cell lines (FIG. 7B).

A2058, SK-MEL-5, SK-MEL-28 (skin), Calu-1, HT-1080, SK-MES-1 (lung), and BXPC3 and HPAFII (pancreas) cells were treated with an MMAE-containing ADC (e.g., anti-p97-MMAE or anti-CD71-MMAE) or Paclitaxel. Supernatant was collected after one night of treatment, and analyzed for HMGB1 release by ELISA. Treatment with MMAE-containing ADC or Paclitaxel elicited HMGB1 release in most of the cell lines assayed (5/7), and was able to induce a more robust response than Paclitaxel in all cell lines assayed. Treatment with anti-CD71-MMAE elicited potent HMGB1 release from 2 of 3 skin cell lines. See, e.g., FIG. 70 and additional description below.

In additional experiments, cell lines treated with MMAE will be co-cultured or “fed” to immune cells derived from peripheral blood mononuclear cells (PBMCs) of healthy donors and the effects of the treated cells on immune cell function assessed.

### Example 2-Analysis of Tubulin Disrupting Agents on Immune Cell Activation

In order to determine the effects of tubulin disrupting agents on the ability of tumor cells to induce immune cell activation, cells treated with tubulin disrupting agents and in the process of undergoing ER stress and potential cell death were fed to antigen presenting cells and effects on APC induction measured.

Macrophages were enriched from PBMCs from 2 healthy donors by adhering PBMCs to cell culture-grade plastic. Non-adherent cells were removed 24 hours later, leaving a population of cells enriched for macrophages.

A2058, SK-MEL-5, and SK-MEL-28 (skin) cells were treated with MMAE, vincristine, and Paclitaxel for 24 hours. Cells were washed and harvested, and subsequently co-cultured with the enriched macrophages prepared above. Macrophages were harvested 4 days after co-culture and assayed for immune activation by flow cytometry. The level of antigen-presentation (as measured by frequency of MHCII-expressing cells) was quantified and normalized to macrophages that were co-cultured with untreated tumor cells. MMAE treatment of tumor cells led to the increase in antigen-presentation in 2/3 tumor cell lines that was more robust than Paclitaxel (FIGS. 8A-8C).

Supernatant from the co-culture of macrophages and dying A2058 and SK-MEL-5 tumor cells was harvested 24 hours later and assayed for levels of cytokine and chemokine production by ELISA and normalized to macrophages that were co-cultured with untreated tumor cells. Treatment of tumor cells with MMAE or an MMAE-containing ADC (anti-p97-MMAE) led to the increase of the indicated cytokines and chemokines that was more robust than treatment with Paclitaxel (FIG. 9A-9B). A2058 cells showed an increase in GM-CSF, IFNg, MCP-3, IL-1 RA, IL-7, MIP-1a, MIP-1b compared to untreated cells while SK-MEL-5 cells showed an increase in GM-CSF, INFa2, IFNg, MCP-3, IL-12p70, IL-17A, IL-1b, MCP-1, MIP-1a, MIP-1b).

### Example 3

### Additional Analysis of Antigen Presentation after Treatment with Tubulin Disrupting Agents

Macrophages were enriched from PBMCs from 2 healthy donors by adhering PBMCs to cell culture-grade plastic. Non-adherent cells were removed 24 hours later, leaving a population of cells enriched for macrophages.

BxPC3 and HPAFII (pancreas) cells were treated with MMAE, vincristine, and Paclitaxel for 24 hours. Cells were washed and harvested, and subsequently co-cultured with the enriched macrophages prepared above. Macrophages were harvested 4 days after co-culture and assayed for immune activation by flow cytometry. Level of antigen-presentation (as measured by frequency of MHCII-expressing cells) was quantified and normalized to macrophages that were co-cultured with untreated tumor cells. MMAE and vincristine treatment of tumor cells led to the increase in antigen-presentation in 1/2 tumor cell lines, whereas treatment with Paclitaxel did not lead to changes in macrophage antigen-presentation (FIGS. 10A-10B), BxPC-3 cells were treated with MMAE, vincristine, or paclitaxel for 17 hours and analyzed for ATP secretion. Treatment with all 3 tubulin-binding agents were able to elicit equivalent levels of ATP secretion (FIG. 100).

After 24 hours of treatment with paclitaxel or an MMAE-containing ADC (anti-p97-MMAE), HMGB1 release from BxPC-3 cells was assessed by ELISA. MMAE-driven cell death led to modestly increased HMGB1 release compared to Paclitaxel treatment (FIG. 10D).

Supernatant from the co-culture of macrophages and dying tumor cells was harvested 24 hours later and assayed for levels of cytokine and chemokine production by ELISA and normalized to macrophages that were co-cultured with untreated tumor cells. Treatment of tumor cells with MMAE or anti-p97-MMAE led to the increase of the indicated cytokines and chemokines that was more robust than treatment with Paclitaxel (FIGS. 11A-11B).

Additional cell lines [Calu-1 (lung), HT1080 (fibrosarcoma) and SK-MES-1 (skin)] were tested for levels of antigen presentation after co culture with macrophages as above. Levels of costimulation (as measured by frequency of CD86-expressing macrophages, Calu-1) or antigen-presentation (as measured by frequency of MHCII-expressing macrophages, HT1080 and SK-MES-1) were quantified and normalized to macrophages that were co-cultured with untreated tumor cells. MMAE treatment of tumor cells led to the increase in immune activation in 3/3 tumor cell lines that was more robust than treatment with Paclitaxel (FIGS. 12A-120). Calu-1, HT-1080, and SK-MES-1 cells were treated with an MMAE-containing ADC (anti-p97-MMAE), vincristine, or paclitaxel for 24 hours and analyzed for HMGB1 release by ELISA. Treatment with anti-p97-MMAE elicited potent HMGB1 release from 2 of 3 cell lines (FIGS. 12D-12F).

Supernatant from the co-culture of macrophages and dying tumor cells was harvested 24 hours later and assayed for levels of cytokine and chemokine production by ELISA and normalized to macrophages that were co-cultured with untreated tumor cells as described above. Treatment of Calu-1 cells with MMAE or an MMAE-containing ADC led to the increase of the indicated cytokines and chemokines that was more robust than treatment with Paclitaxel (FIG. 13).

MCF7 (Triple Negative Breast Cancer) cells were treated with MMAE, an MMAE-containing ADC (anti-CD71 OKT9-1006), vincristine, and Paclitaxel for 24 hours. Cells were washed and harvested, and subsequently co-cultured with the enriched macrophages prepared above. Macrophages were harvested 4 days after co-culture and assayed for immune activation by flow cytometry. Treatment of tumor cells with MMAE or an MMAE-containing ADC led to the increase in macrophage antigen presentation, as measured by frequency of MHCII-expressing macrophages, that was more robust than treatment with Paclitaxel (FIG. 14A). Supernatant from the co-culture of macrophages and dying MCF7 cells was harvested 24 hours later and assayed for levels of cytokine and chemokine production by ELISA and normalized to macrophages that were co-cultured with untreated tumor cells. Treatment of MCF7 cells with MMAE or an MMAE-containing ADC led to the increase of the indicated cytokines and chemokines that was more robust than treatment with Paclitaxel (FIG. 14B).

MCF7 cells were treated with MMAE or an MMAE-containing ADC (SGN-LIV1A) for 16 hours and harvested in RPA buffer for western bot analysis. Treatment with MMAE activated all 3 pathways of the ER stress response, as indicated by phosphorylation of IRE1 and eIF2a, as well as cleavage of full-length ATF6. Severe ER stress is a prerequisite to the exposure of pro-phagocytic signals on the surface of tumor cells, and is elicited by MMAE as indicated by activation of JNK signaling by phosphorylated IRE1, and expression of CHOP (FIG. 15A). Induction of ICD is also characterized by the secretion of ATP and HMGB1. Extracellular ATP serves as a strong chemotactic signal, promoting immune cell migration to the tumor site. Upon arrival, extracellular HMGB1 signals through various pro-inflammatory receptors (TLR2, TLR4, RAGE) to activate antigen-presenting cells, thereby promoting immune activity within the tumor. Treatment of MCF7 cells with MMAE and SGN-LIV1A leads to increased secretion of ATP and HMGB1 (FIGS. 15B-150).

Treatment of MCF7 with SGN-LIV1A or eribulin elicits severe ER stress (phosphorylation of IRE1 and JNK), whereas Paclitaxel and Docetaxel do not elicit JNK phosphorylation. Increased AIR secretion was also evident after 48 hours of treatment with SGN-LIV1A, or eribulin, indicating potent ICD induction as a result of microtubule disruption, whereas neither Paclitaxel nor Docetaxel elicited ATP secretion (FIGS. 16A-16B).

SOD mice were subcutaneously engrafted with MCF7 cells. Upon reaching 200 cubic millimeters, mice received a single intraperitoneal dose of an MMAE-containing ADC (SGN-LIV1A or anti-CD71-MMAE). 8 days post-ADC treatment, tumors were excised and assessed for immune cell composition by flow cytometry. As a result of MMAE-driven cell death and immunogenicity, MMAE-ADC treated tumors showed increased level of immune activation by tumor-infiltrating immune cells (FIGS. 17A-17E).

MDA-MB-468 cells were treated with MMAE or an MMAE-containing ADC (ASG-22ME). Supernatant was collected after 48 hours of treatment, and analyzed for ATP secretion. Treatment with MMAE or ASG-22ME elicited robust ATP secretion that was comparable to thapsigargin, a known ER stress and autophagy inducer (FIG. 18).

Liver tumor lines Hep3b, Huh7, and JHH7 cells were treated with MMAE, Tubulysin M, vincristine, and Paclitaxel for 24 hours and analyzed for ATP secretion. Treatment with MMAE or Tubulysin M elicited potent ATP secretion from 3 of 3 cell lines evaluated (FIGS. 19A-C). Cells were washed and harvested, and subsequently co-cultured with enriched macrophages prepared as above. Macrophages were harvested 4 days after co-culture and assayed for immune activation by flow cytometry. Levels of costimulation (as measured by CD86 expression, JHH7) and antigen-presentation (as measured by frequency of MHCII-expressing cells, Hep3b, Huh7, and JHH7) were quantified and normalized to macrophages that were co-cultured with Paclitaxel-treated tumor cells. MMAE treatment of tumor cells led to the increase in immune-activation in 3/3 tumor cell lines that was more robust than Paclitaxel (FIG. 19D-19G). Supernatant from the co-culture of macrophages and dying liver tumor cells was harvested 24 hours later and assayed for levels of cytokine and chemokine production by ELISA and normalized to macrophages that were co-cultured with untreated tumor cells. Treatment of Hep3b, Huh7, and JHH7 cells with MMAE or Tubulysin M led to the increase of the indicated cytokines and chemokines that was more robust than treatment with Paclitaxel (FIG. 20A-200).

T-24 (bladder) cells were treated with MMAE or an MMAE-containing ADC (ASG-22ME) for 48 hours and analyzed for ATP secretion. Treatment with MMAE or ASG-22ME elicited potent ATP secretion (FIG. 21A). T-24 cells were also treated with MMAE, an MMAE-containing ADC (ASG22ME, Enfortumab vedotin), vincristine, and Paclitaxel for 24 hours. Cells were washed and harvested, and subsequently co-cultured with the enriched macrophages as above. Macrophages were harvested 4 days after co-culture and assayed for immune activation by flow cytometry. Level of antigen-presentation (as measured by frequency of MHCII-expressing macrophages) was quantified and normalized to macrophages that were co-cultured with untreated tumor cells. Treatment of T-24 cells with MMAE or an MMAE-containing ADC led to the increase in macrophage antigen presentation that was more robust than treatment with Paclitaxel (FIG. 21B). Supernatant from the co-culture of macrophages and dying T-24 tumor cells was harvested after 24 hours and assayed for levels of cytokine and chemokine production by ELISA and normalized to macrophages that were co-cultured with untreated tumor cells. Treatment of T-24 cells with MMAE or an MMAE-containing ADC led to the increase of the indicated cytokines and chemokines that was more robust than treatment with Paclitaxel (FIG. 210).

U-266 multiple myeloma cells were treated with free MMAE (492 nM), an MMAE-containing ADC (SGN-CD48A, 10 ng/ml), and a non-binding MMAE-ADO (10 ng/ml) for 24 and 48 hours. Cells were harvested at each time point and whole cell lysates were prepared. Lysates for each sample were run on an SDS-Page and transferred onto a nitrocellulose membrane. Western blot analysis was performed using phospho-JNK Thr183/Tyr185 (pJNK), PARR, ATF4, AT6, phospho-IRE-1 Ser274 (pIRE-1). β-actin serves as a loading control. Multiple myeloma cells such as U-266 exhibit high levels of endogenous ER stress indicated by detection of basal pJNK and pIRE-1 staining (FIG. 22A). However, treatment with MMAE and SGN-CD48A, further increased the ER stress response, as indicated by the elevation in ATF4 expression as well as phosphorylation of MK The cleavage of PARP (lower molecular weight band) is an indicator of cells undergoing apoptosis. Importantly, the induction of ER stress by MMAE in U-266 cells was sufficient to elicit markers of ICD.

U-266 cells were treated for 48 hours with free MMAE (492 nM), an MMAE-containing ADC (SGN-CD48A, 10 ng/ml), and a non-binding MMAE-ADC (10 ng/ml) for 48 hours. Cells were harvested, washed in flow buffer, and subsequently stained for both AnnexinV, a marker for apoptosis, and HSP70. Cells were also stained with both AnnexinV and calreticulin. In both conditions, cells that were AnnexinV negative were selected and the percent population that was positive for HSP70 or calreticulin were determined. An increase in the percentage of ICD markers are evident on the cell surface upon treatment with SGN-CD48A and free MMAE prior to death (FIG. 22B), providing potent pro-phagocytic signals to enhance anti-tumor immunity.

Numerous modifications and variations of the invention as set forth in the above illustrative examples are expected to occur to those skilled in the art. Consequently only such limitations as appear in the appended claims should be placed on the invention.

1. (canceled)

2. A method for modulating ATP release in a solid tumor comprising administering to a subject in need thereof an antibody drug conjugate agent having the formula Drug-Linker Unit-Antibody (D-LU-Ab), wherein D is a tubulin disrupting agent, in an amount effective to induce apoptosis in the solid tumor.

3. A method of inducing immune cell migration to a solid tumor comprising administering to a subject in need thereof an antibody drug conjugate agent having the formula Drug-Linker Unit-Antibody (D-LU-Ab), wherein D is a tubulin disrupting agent, in an amount effective to induce immune cell infiltration into the solid tumor.

4. A method for inducing immunogenic cell death (ICD) in a solid tumor comprising administering to a subject in need thereof an antibody drug conjugate agent having the formula Drug-Linker Unit-Antibody (D-LU-Ab), wherein D is a tubulin disrupting agent, in an amount effective to induce immunogenic cell death in the solid tumor.

5. The method of claim 2 wherein the antibody is specific for CD30, CD19, CD70, CD71, CD20, CD52, CD133, EGFR, HER2, VEGF, VEGFR2, PD-1, PDL1, RANKL, CTLA-4, IL-6, SLAMF7, CD3, TNF-alpha, PDGFR-alpha, CD38, GD2, cCLB8, p97, Nectin-4, or EpCAM.

6. The method of claim 2 wherein the tubulin-disrupting agent increases ER stress protein pathways, increases ATP secretion and increases High mobility group box 1 (HMGB1) protein.

7. The method of claim 2 wherein the tubulin disrupting agent is selected from the group consisting of an auristatin, a tubulysin, a colchicine, a vinca alkaloid, a taxane, a cryptophycin, a maytansinoid, a hemiasterlin, and other tubulin disrupting agents.

8. The method of claim 2 wherein the tubulin disrupting agent is an auristatin selected from the group consisting of monomethyl auristatin E (MMAE) monomethyl auristatin F (MMAF), and dolostatin-10.

9. The method of claim 2 wherein the tubulin disrupting agent is a tubulysin selected from the group consisting of tubulysin D, tubuphenylalanine and tubutyrosine.

10. The method of claim 2 wherein the tubulin disrupting agent is a colchicine selected from the group consisting of colchicine and CA-4.

11. The method of claim 2 wherein the tubulin disrupting agent is a vinca alkaloid selected from the group consisting of Vinblastine (VBL), vinorelbine (VRL), vincristine (VCR) and vindesine (VDS).

12. The method of claim 2 wherein the tubulin disrupting agent is a taxane selected from the group consisting of paclitaxel and docetaxel.

13. The method of claim 2 wherein the tubulin disrupting agent is a cryptophycin selected from the group consisting of cryptophycin-1 and cryptophycin-52.

14. The method of claim 2 wherein the tubulin disrupting agent is a maytansinoid selected from the group consisting of maytansine, maytansinol, maytansine analogs, DM1, DM3 and DM4, and ansamatocin-2.

15. The method of claim 2 wherein the tubulin disrupting agent is a hemiasterlin selected from the group consisting of hemiasterlin and HTI-286.

16. The method of claim 2 wherein the tubulin disrupting agent is selected from the group consisting of taccalonolide A, taccalonolide B, taccalonolide AF, taccalonolide AJ, taccalonolide AI-epoxide, discodermolide, epothilone A, epothilone B, and laulimalide.

17. The method of claim 2 wherein the solid tumor is selected from the group consisting of lung cancer, breast cancer, ovarian cancer, cervical cancer, gastrointestinal cancers, head and neck cancer, melanoma, sarcoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm, renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non small cell lung cancer (NSCLC), squamous cell non-small cell lung cancer, breast cancer, recurrent metastatic breast cancer, hepatocellular carcinoma, richter's syndrome; waldenstrom macroglobulinemia, adult glioblastoma; adult gliosarcoma, recurrent glioblastoma, recurrent childhood rhabdomyosarcoma, recurrent ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and Sezary syndrome.

18. The method of claim 2 wherein the antibody drug conjugate comprises a protease cleavable linker, an acid-cleavable linker or a disulfide linker.

19. The method of claim 18 wherein the protease cleavable linker comprises a thiolreactive spacer and a dipeptide.

20. The method of claim 18, wherein the protease cleavable linker consists of a thiolreactive maleimidocaproyl spacer, a valine-citrulline dipeptide, and a p-amino-benzyloxycarbonyl spacer.

21. The method of claim 18 wherein the acid cleavable linker is a hydrazine linker or a quaternary ammonium linker.

22. The method of claim 2 further comprising administering a chemotherapy regimen.

23. The method of claim 22 wherein the chemotherapy regimen consists essentially of
i) doxorubicin, vinblastine, and dacarbazine (AVD) as a combination therapy; or
ii) cyclophosphamide, vincristine and prednisone (CHP) as a combination therapy.

24. (canceled)

25. The method of claim 2 wherein the antibody of the antibody drug conjugate is a monoclonal antibody.

26. The method of claim 2 wherein the antibody is an anti-CD30 antibody and the anti-CD30 antibody drug conjugate comprises
i) a heavy chain CDR1 set out in SEQ ID NO: 4, a heavy chain CDR2 set out in SEQ ID NO: 6, a heavy chain CDR3 set out in SEQ ID NO: 8; and
ii) a light chain CDR1 set out in SEQ ID NO: 12, a light chain CDR2 set out in SEQ ID NO: 14, and a light chain CDR13 set out in SEQ ID NO: 16.

27. The method of claim 2 wherein the antibody is an anti-CD30 antibody and the anti-CD30 antibody drug conjugate comprises
i) an amino acid sequence at least 85% identical to a heavy chain variable region set out in SEQ ID NO: 2 and
ii) an amino acid sequence at least 85% identical to a light chain variable region set out in SEQ ID NO: 10.

28. The method of claim 2 wherein the antibody is an anti-CD30 antibody and the anti-CD30 antibody of the antibody drug conjugate is a chimeric AC10 antibody.

29. The method of claim 2 wherein the antibody drug conjugate comprises monomethyl auristatin E and a protease-cleavable linker.

30. (canceled)

31. The method of claim 29, wherein the protease cleavable linker consists of a thiolreactive maleimidocaproyl spacer, a valine-citrulline dipeptide, and a p-amino-benzyloxycarbonyl spacer.

32. The method of claim 2 wherein the anti-CD30 antibody drug conjugate is brentuximab vedotin.

33. The method of claim 2 wherein the antibody drug conjugate induces immune cell migration to the site of the tumor.


SEQUENCE LISTING (16 sequences)

SEQ ID NO: 1 - DNA, 351 bp, Mus musculus
  source 1..351: mol_type = unassigned DNA
  CDS 1..351: protein_id = 2
cagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgca

SEQ ID NO: 2 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 3 - DNA, 15 bp, Mus musculus
  source 1..15: mol_type = unassigned DNA
gactactatataacc

SEQ ID NO: 4 - protein, 5 aa, Mus musculus
DYYIT

SEQ ID NO: 5 - DNA, 51 bp, Mus musculus
  source 1..51: mol_type = unassigned DNA
tggatttatcctggaagcggtaatactaagtacaatgagaagttcaagggc

SEQ ID NO: 6 - protein, 17 aa, Mus musculus
WIYPGSGNTKYNEKFKG

SEQ ID NO: 7 - DNA, 24 bp, Mus musculus
  source 1..24: mol_type = unassigned DNA
tatggtaactactggtttgcttac

SEQ ID NO: 8 - protein, 8 aa, Mus musculus
YGNYWFAY

SEQ ID NO: 9 - DNA, 333 bp, Mus musculus
  source 1..333: mol_type = unassigned DNA
  CDS 1..333: protein_id = 10
gacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 10 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 11 - DNA, 45 bp, Mus musculus
  source 1..45: mol_type = unassigned DNA
aaggccagccaaagtgttgattttgatggtgatagttatatgaac

SEQ ID NO: 12 - protein, 15 aa, Mus musculus
KASQSVDFDGDSYMN

SEQ ID NO: 13 - DNA, 21 bp, Mus musculus
  source 1..21: mol_type = unassigned DNA
gctgcatccaatctagaatct

SEQ ID NO: 14 - protein, 7 aa, Mus musculus
AASNLES

SEQ ID NO: 15 - DNA, 27 bp, Mus musculus
  source 1..27: mol_type = unassigned DNA
cagcaaagtaatgaggatccgtggacg

SEQ ID NO: 16 - protein, 9 aa, Mus musculus
QQSNEDPWT